(19) United States
(12) Patent Application Publication
(10) Pub. No.: US 2008/0260902 A1
(43) Pub. Date: Oct. 23, 2008

(54) Process for Increasing Throughput of Corn for Oil Extraction

(76) Inventors: Michael Van Houten, Carmel, IN (US); Michael J. Beaver, Victoria, MN (US); Aharon M. Eyal, Jerusalem (IL); Eugene J. Fox, Dayton, OH (US); Joel Ingvalson, Minneapolis, MN (US); Neal T. Jakel, Janesville, WI (US); Douglas C. Kotowski, St. Louis Park, MN (US); Paul J. McWilliams, Racine, WI (US); Alexander Patist, Shakopee, MN (US); Michael J. Tupy, Crystal, MN (US); Troy T. Lohrmann, Lake Villa, IL (US)

Correspondence Address: SENNIGER POWERS LLP, ONE METROPOLITAN SQUARE, 16TH FLOOR, ST LOUIS, MO, 63102, US

(21) Appl. No.: 11/587,258

(22) PCT Filed: Apr. 14, 2005
(86) PCT No.: PCT/US2005/012545

§ 371 Date: Apr. 22, 2008

Related U.S. Application Data
(60) Provisional application No. 60/564,202, filed on Apr. 21, 2004.
(60) Provisional application No. 60/628,069, filed on Nov. 15, 2004.

(51) Int. Cl. A23L 1/105; A23D 9/00; A23L 1/10; C10L 5/44

(52) U.S. Cl. 426/18; 426/417; 426/622; 44/605

(57) ABSTRACT

Corn oil is extracted from corn to form a corn meal. Processing the corn grain to obtain the oil, meal, and other product streams generally includes dividing the corn kernel by fractionating to create a higher oil fraction and a lower oil fraction, forming a solvent extractable structure from the higher oil fraction, and extracting the oil from the higher oil fraction. The extracted corn oil is useful for making nutritionally enhanced edible oil or cooking oil, lubricants, biodiesel, fuel, cosmetics and oil-based or oil-containing chemical products. The extracted corn meal is useful for making enhanced animal feed rations, snack food, blended food products, cosmetics, and fermentation broth additive. The lower oil fraction is useful for one or more processes such as fermentation, wet-milling, animal feed production, sweetener production, and starch production, making enhanced animal feed rations, snack food, blended food products, and cosmetics.

### CROSS-REFERENCE TO RELATED APPLICATIONS

This application claims the benefit of U.S. Provisional Application No. 60/564,202, filed Apr. 21, 2004 and U.S. Provisional Application No. 60/628,069 filed Nov. 15, 2004, which are herein incorporated by reference.

### BACKGROUND OF THE INVENTION

The present invention relates to a process for increasing the output of extracted corn oil.

Corn, Zea mays, is grown for many reasons including its use in food and industrial applications. Corn oil and corn meal are two of many useful products derived from corn. Commercial processing plants utilizing conventional methods for extracting corn oil from conventional corn separate the corn seed into its component parts, e.g., endosperm, germ, tip cap, and pericarp, and then extract corn oil from the corn germ fraction. Corn germ produced by wet or dry milling may be processed by pressing the germ to remove the oil or by flaking the germ, pre-pressing, and extracting the oil with a solvent. In both of these processes, because the germ was separated from the remainder of the kernel, many or all of the valuable components of the endosperm fraction are absent from the oil.

In contrast to the traditional wet or dry milling of the separated corn germ, other processes involve the whole corn kernel, resulting in an increase in the oil of the components from the endosperm. U.S. Pat. Nos. 6,313,328 and 6,388,110 describe a commercial-scale method for processing whole kernel corn grain having a total oil content of at least about 8 wt. %, including the steps of flaking corn grain and extracting a corn oil from the flaked corn grain. The method can be effectuated by processing the corn grain using methods and equipment typically used to process soybeans and other similar oilseed types. U.S. Pat. No. 6,610,867 describes a process for extracting corn oil to form corn meal. The process generally includes the steps of cracking whole kernel corn having a total oil content of from about 3 wt. % to about 30 wt. % and extracting a corn oil from the cracked corn grain. The corn is not flaked. U.S. Pat. No. 6,648,930 discloses products comprising extracted corn oil and corn meal obtained from whole high oil corn. U.S. Patent Publication No. 2002/0151733A1 discloses methods of manufacturing and processing corn oil and corn meal by flaking whole corn grain having a total oil content of from about 3 wt. % to about 6 wt. % and extracting a corn oil from the flaked corn grain.

### BRIEF SUMMARY OF THE INVENTION

The present invention includes a method of dividing a whole corn kernel of high oil corn comprising: fractionating a whole corn kernel of high oil corn having a range of moisture from about 8 wt. % to about 22 wt. %, and further having an endosperm component and a germ component, into a higher oil fraction and a lower oil fraction, wherein, the higher oil fraction has an oil concentration greater than that of the corn kernel and the lower oil fraction has an oil concentration less than that of the corn kernel. In one embodiment, fractionating comprises contacting the whole corn kernel with an abrasive screen to separate at least a portion of the germ component of the corn kernel from at least a portion of the remainder of the corn kernel. In one embodiment, fractionating comprises subjecting the whole corn kernel to a Buhler L machine, a Satake debranner, or other means for contacting the corn kernel with a device to remove at least a portion of the germ component of the corn kernel from at least a portion of the remainder of the corn kernel. In one embodiment, the method further comprises separating the lower oil fraction into larger and smaller pieces of lower oil fraction and fractionating the larger pieces of the lower oil fraction into a second stage higher oil fraction and a second stage lower oil fraction. Optionally, the second stage higher oil fraction and the higher oil fraction are combined. Optionally, the second stage lower oil fraction and the lower oil fraction are combined.

Another embodiment of the present invention includes cracking the corn kernel into at least two differing sized pieces of cracked corn prior to fractionating the cracked corn. In one embodiment, cracking comprises cutting the endosperm component of the corn material into pieces of predominantly from about 2540 microns to about 4270 microns in size and producing a germ component predominantly greater than about 4750 microns in size. In another embodiment, cracking step comprises using a corrugated roller mill.

In one embodiment, a first size of cracked corn pieces comprise predominantly small size pieces of cracked corn which comprise less than about 10 wt. % of the cracked corn pieces. In one embodiment, the small size pieces of cracked corn are less than about 1080 microns in size. In one embodiment, a second size of cracked corn pieces comprise medium size pieces of cracked corn which comprise about 70 wt. % of the cracked corn pieces. In one embodiment, the second size pieces of cracked corn are from about 2540 to 4270 microns in size. In one embodiment, a third size of cracked corn pieces comprise large pieces of cracked corn which comprise about 20 wt. % of the cracked corn pieces. In one embodiment, the third size pieces of cracked corn predominantly are greater than about 4750 microns in size. In one embodiment, third size of cracked corn pieces comprises about 30 wt. % to about 40 wt. % germ component. In another embodiment, at least three sizes of cracked corn pieces are produced and the large size pieces of cracked corn comprise from about 11 wt. % to about 22 wt. % oil and the small and medium size pieces of cracked corn comprise about 4.5 wt. % to about 8 wt. % oil. In another embodiment, the large size pieces of cracked corn comprise about 16% wt. % oil.

In an alternative embodiment, a portion of the cracked corn pieces is separated into at least two fractions according to their size. Suitable separating techniques include size separation or gravity separation. One size separation technique comprises screening.

In one embodiment, the method comprises fractionating a portion of the small size pieces of cracked corn material into a higher oil fraction and a lower oil fraction, wherein the higher oil fraction has an oil concentration greater than that of the small size pieces of cracked corn material and the lower oil fraction has an oil concentration less than that of the small size pieces of cracked corn material. In one embodiment, a portion of the smaller size pieces of cracked corn are aspirated to remove bran. In one embodiment, a portion of the larger size pieces of cracked corn are flaked or ground.

In a further embodiment, the corn kernel, the cracked corn pieces, and/or the higher oil fraction of the cracked corn pieces are tempered at a temperature and for a time sufficient to increase the differential hardness between the germ component and the remainder of the corn kernel. In one embodiment, the corn kernel or cracked corn pieces are tempered up to a maximum of about 1% additional moisture, about 2% additional moisture, or about 3% additional moisture. In one embodiment, tempering comprises heating the corn material directly or indirectly and adding moisture to the corn material by spraying water, an aqueous solution, and/or sparging steam.

In an alternative embodiment of the present invention, the oil is extracted from a portion of the higher oil fraction, combination corn material, flaked and cracked corn, or ground and cracked corn, from a first combination material comprising a portion of the ground cracked corn and a portion of the higher oil fraction, from a second combination material comprising a portion of the first combination material and a portion of the flaked corn material, from a third combination material comprising a portion of the flaked, cracked corn and a portion of the higher oil fraction to produce an extracted corn oil and an extracted corn meal. One of skill in the art will appreciate that other corn material, or other corn material containing oil, can also be added to a product produced in the present invention, and the oil extracted.

In one embodiment, a portion of the higher oil fraction, combination material, or ground, cracked corn, or first combination material is formed into a solvent-extractable structure. Methods used in forming the solvent-extractable structure include one or more of extruding, expanding, expelling, pelleting or enzymatic treatment. Solvent extraction is one method of extracting oil from a portion of the solvent-extractable structure to produce an extracted corn oil and an extracted corn meal. Useful solvents for solvent extraction include, for example, hydrocarbons, alkanols, alkanol-containing aqueous solutions, and supercritical carbon dioxide. Examples of such solvents include, but are not limited to C₂-C₈ hydrocarbons, C₁-C₄ alkanols, including methanol, ethanol and isopropanol. Mixtures of solvents may be used. Hexane(s) is a preferred solvent. In one embodiment, the solvent comprises carbon dioxide from a fermentation process.

In one embodiment, a portion of the extracted corn meal or extracted corn oil is desolventized.

The whole high oil corn kernel comprises from at least about 3.5 wt. %, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 9.5%, at least about 10%, at least about 10.5%, at least about 11%, at least about 11.5%, at least about 12%, at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20%, at least about 20.5%, at least about 21%, at least about 21.5%, to about 22 wt. % oil on a dry matter basis. In one embodiment, the corn kernel comprises at least about 3.5 wt. % oil on a dry matter basis.

In one embodiment, the lower oil fraction comprises less than about 3 wt. % oil on a dry matter basis. The range of moisture in the whole corn kernel of high oil corn is from about 8% to about 18%.

The invention further comprises using a portion of the lower oil fraction as a feedstock for fermentation, wet corn milling, food, pet food or other process. In another embodiment, the invention further comprises using a portion of the desolventized, extracted meal as a feedstock for fermentation, wet corn milling, food, pet food, or other process.

In one embodiment, the invention comprises using a portion of the higher oil fraction as a feedstock for fermentation, food, pet food, or other process. In an alternative embodiment, the invention comprises using a portion of removed bran as a feedstock for extraction. In another embodiment, the invention comprises extracting a portion of phytosterols from the bran feedstock.

In another embodiment of the present invention, feed pellet quality is improved by substituting Enhanced Meal of the present invention for yellow #2 corn. In one embodiment, the substitution of Enhanced Meal for yellow #2 corn provides for decreased energy usage at the feed mill.

Another aspect of the invention provides methods of separating whole kernel corn into a high oil fraction using a degerminator that provides a higher oil fraction wherein less than 50%, 60%, 70%, 80% or 90% of the germ in the high oil fraction is intact. Exemplary degerminators include the Buhler-L apparatus (Buhler GmbH, Germany), a Satake VCW debranning machine (Satake USA, Houston, Tex.), or other apparatus wherein the incoming corn material is contacted with an abrasive device such as a screen to remove the hull and the germ component of the corn material from a portion of the remainder of the corn material (endosperm component). The lower oil fraction from machines using abrasive forces, such as those mentioned herein produces a lower oil fractions and high oil fractions that are useful as a fermentation feedstocks and/or streams that can be introduced into corn wet milling processes. When the low oil fraction and/or the higher oil fraction is used as a fermentation feedstock or as a stream that is introduced into a corn wet milling process the starting material can be any variety of whole kernel corn, including yellow #2 dent as well as higher oil corn.

The present invention relates to products that are derived from oil and meal extracted from corn pursuant to the process of the present invention, and the uses of such products, including, but not limited to, the extracted corn oil produced by any of the methods of the present invention, extracted corn meal (whether or not desolventized) produced by any of the methods of the present invention, an animal feed comprising such meal, the lower oil fraction produced by any of the methods of the present invention, an animal feed comprising such lower oil fraction, and an animal feed comprising the combination of such an extracted corn meal and a lower oil fraction. In addition, the present invention relates to the use of such products, including the use of such products in food and other products more fully described herein.

### BRIEF DESCRIPTION OF THE SEVERAL VIEWS OF THE DRAWING(S)

FIG. 1 is a schematic flow chart of one embodiment of the present invention.

FIG. 2 is a schematic flow chart of one embodiment of a two stage fractionation process of the present invention.

FIG. 3 is a front elevational view of a fractionating apparatus with a six-sided screen.

FIG. 4 is a cross section of a polygonal-sided screen of a fractionating apparatus.

FIGS. 5, 5A, and 5B are schematic flow charts of alternative embodiments of the present invention.

FIG. 6 is a schematic flow chart of an alternative embodiment of the present invention.

### DETAILED DESCRIPTION OF THE INVENTION

Botanically, a maize kernel is known as a caryopsis, a dry, one-seeded, nut-like berry in which the fruit coat and the seed are fused to form a single grain. Mature kernels are composed of four major parts: pericarp (hull or bran), germ (embryo), endosperm, and tip cap.

The scutellum and the embryonic axis are the two major components of the germ. The scutellum makes up about 90% of the germ and stores nutrients mobilized during germination. During germination, the embryonic axis grows into a seedling. The germ is characterized by its high fatty oil content. It is also rich in crude proteins, sugars, and ash constituents. The scutellum contains oil-rich parenchyma cells which have pitted cell walls.

The endosperm contains the starch, and is lower in protein than the germ and the bran. It is also low in crude fat and ash constituents. Corn endosperm includes some valuable components such as carotenoids, lutein, and zeaxanthin. Carotenoids in grains are classified into two general groups, the carotenes and the xanthophylls. The carotenes are important because they are vitamin A precursors. Blessin et al. (Cereal Chemistry, 40, 582-586 (1963)) found that over 90% of the carotenoids, of which beta-carotene is predominant, are located in the endosperm of yellow dent corn and less than 5% are located in the germ. Vitamin A is derived primarily from beta-carotene. Another group of valuable components found in the endosperm includes the tocotrienols. Grams et al. (1970) discovered that in corn, tocotrienols were found only in the endosperm, whereas the germ contained most of the tocopherols. Tocotrienols can be extracted from plant material using various solvents. Processes for recovering tocotrienols from plant material are described by Lane et al. in U.S. Pat. No. 5,908,940, the entire disclosure of which is incorporated by reference. Accordingly, the process described herein provides a nutritionally enhanced corn oil enriched with lutein, zeaxanthin, and/or beta-carotene and optionally one or more other nutritional components. Oil-based products made with corn oil obtained by a process of the present invention described herein can contain higher levels of important nutrients than similar products made with corn oil produced by conventional methods. The corn oil obtained by the extraction methods described herein will include the corn oil from the both germ component and endosperm component, and may include one or more other components extracted from the rest of the kernel. The one or more other components can be oil from the endosperm, tocotrienols, tocopherols, carotenoids, carotenes, xanthophylls, and sterols. Tocopherols (vitamin E) and vitamin A are antioxidants and fat-soluble vitamins. When included in the diet, both have demonstrated health benefits. Blending of oil of the present invention with other oils or substances to achieve an appropriate level of beta-carotene, vitamin E, and tocotrienols is deemed within the scope of the present invention. In some embodiments, extracted corn oil prepared as described herein comprises about 0.1 wt. % to about 0.5 wt. % of tocopherol. Oil produced in accordance with the present invention also may include approximately a 200% to 300% increase in tocotrienol content over conventionally-produced crude corn oil. Using a method of the present invention, the corn is prepared and corn oil extracted, and then analyzed for tocotrienol content. The actual minimum and maximum values for tocotrienol content will depend upon the particular high oil corn used.

Extraction of carotenes and xanthophylls and other pigments is described in detail by Blessin (Cereal Chemistry, 39, 236-242 (1962); the entire disclosure of which is incorporated by reference). Combinations of solvents, primarily ethanol and hexanes, can be used to extract carotenes and xanthophylls from corn. Ethanol, hexanes, other solvents combinations, and ratios thereof may be used to produce oil of the present invention on a commercial scale.

Exemplary embodiments of the crude oil obtained according to the extraction method described herein generally possess the partial composition profile featured in Table 1.

### Table 1

TABLE 1

| | Exemplary Extracted | Extracted High Oil |
|---|---|---|
| Component | High Oil Corn | Corn (Range) |
| FFA (%) | 1.45 | 0.7-3.00 |
| C16:0 | 11.4 | 10-14 |
| C18:0 | 2.1 | 1.5-3.5 |
| C18:1, cis | 33 | 26-50 |
| C18:1, trans | | |
| C18:2, cis | 50 | 42-60 |
| C18:2, trans | | |
| C18:3 | 0.8 | 0.6-1.6 |
| Phosphorus (ppm) | 190 | 100-400 |
| Total Tocopherols (%) | 0.13 | 0.1-.50 |

Fatty acids generally found in the corn oil generally include palmitic, stearic, oleic, linoleic, and linolenic acids.

The maize kernel is covered by a water-impermeable cuticle. The pericarp is the mature ovary wall which is beneath the cuticle, and comprises all the outer cell layers down to the seed coat. It is high in non-starch-polysaccharides, such as cellulose, pentosans, and hemicellulose. Because of its high fiber content, the pericarp is tough. The tip cap, where the kernel is joined to the cob, is a continuation of the pericarp, and is usually present during shelling. It contains a loose and spongy parenchyma.

Whole kernel corn seed or grain harvested from any of several different types of corn plants can be used in the present invention. These types of corn plants are, for example, hybrids, inbreds, transgenic plants, genetically modified plants, or a specific population of plants. Useful corn grain types include, for example, flint corn, popcorn, flour corn, dent corn, white corn, and sweet corn. As used herein, the terms “whole kernel” or “whole corn” mean a kernel that has not been separated into its constituent parts, e.g. the hull, endosperm, tip cap, pericarp, and germ have not been purposefully separated from each other. Purposeful separation of one corn constituent from another does not include random separation that may occur during storage, handling, transport, crushing, flaking, cracking, grinding, or abrading. A purposeful separation of the constituent part is one wherein at least 50% of one constituent, e.g., germ, has been separated from the remaining constituents. As used herein, the term “corn material” refers to whole corn, cracked corn, screened corn, and aspirated corn, whether or not conditioned or tempered.

Useful corn grain for the processing method of the present invention has a total oil content from at least about 3.5 wt. % to at least about 22 wt. % on a dry matter basis. The total oil content of corn grain suitable for the present invention can be, for example, grain having an oil content at least about 3.5 wt. %, at least about 5 wt. %, at least about 6 wt. %, at least about 7 wt. %, at least about 8 wt. %, at least about 9 wt. %, at least about 11 wt. %, at least about 12 wt. %, at least about 15 wt. %, at least about 18 wt. %, at least about 20 wt. %, at least about 22 wt. %, from about 3.5 wt. % to about 22 wt. %, from about 10 wt. % to about 22 wt. %, or from about 14 wt. % to about 22 wt. %, and values within those ranges.

Although the oil content can be determined at any moisture content, it is acceptable to normalize the oil content to a moisture content of about 15.5%.

Preferably, the corn grain used in the process of the present invention is high oil corn. As used herein, the phrase “high oil corn” refers to corn grain comprising at least about 6 wt. % or greater, preferably at least about 7 wt. % or greater, and preferably at least about 8 wt. % or greater oil. A high oil corn has an elevated level of oil as compared to conventional yellow dent corn, which has an oil content of about 3 wt. % to about 5 wt. %. High oil corn useful in making the oil and meal described herein are available from Pfister Hybrid Corn Co. (El Paso, Ill.), Wyffels Hybrids Inc. (Geneseo, Ill.), Galilee Seeds Research & Development (Rosh Pina, Israel), or DuPont Specialty Grains (Johnston, Iowa). Other suitable high oil corn includes the corn populations known as Illinois High Oil (IHO) and Alexander High Oil (Alexo), samples of which are available from or through the University of Illinois Maize Genetics Cooperation Stock Center (Urbana, Ill.). Methods for developing corn inbreds, hybrids, transgenic species, and populations that generate corn plants producing grain having elevated oil concentrations are known and described in Lambert (Specialty Corn, CRC Press Inc., Boca Raton, Fla., pp. 123-145 (1994) and in U.S. Patent Publication No. 2003/0182697, incorporated herein by reference.

Corn grain having an elevated total oil content is identified by any of a number of methods known to those of ordinary skill in the art. The oil content of grain, including the fat content of a meal extracted from the grain, can be determined using American Oil and Chemical Society Official Method, 5^{th} edition, March 1998, (“AOCS method Ba 3-38”). AOCS method Ba 3-38 quantifies substances that are extracted by petroleum ether under conditions of the test. The oil content or concentration is the weight percentage of the oil with respect to the total weight of the seed sample. Oil content may be normalized and reported at any desired moisture basis. Other suitable methods for identifying high oil corn grain include using a near infrared (NIR) oil detector to select corn ears having corn kernels with elevated oil levels. Likewise, an NIR detector can also be used to select individual corn kernels having elevated levels of corn oil. However, selecting individual ears and/or kernels having elevated oil content may not be cost effective in identifying high oil kernels suitable for processing using methods described herein. Generally, corn seed producing corn plants that yield grain having elevated total oil concentrations is planted and harvested using known farming methods.

Generally, the range of hardness of the commercial corn grain used in the present invention is in the range of from about 46 to about 60 wt. % as measured by a Quaker hardness test; but corn grains having a hardness outside of this range may also be used. A preferred grain hardness is 55 wt. %. Altering the hardness of the grain will cause changes to the operating conditions to obtain the best results. For example, the harder the grain, the more heat that will be used to temper the grain. Harder grains provide better feed for cracking and screening processes. However, softer grains increase throughput characteristics at the fractionator.

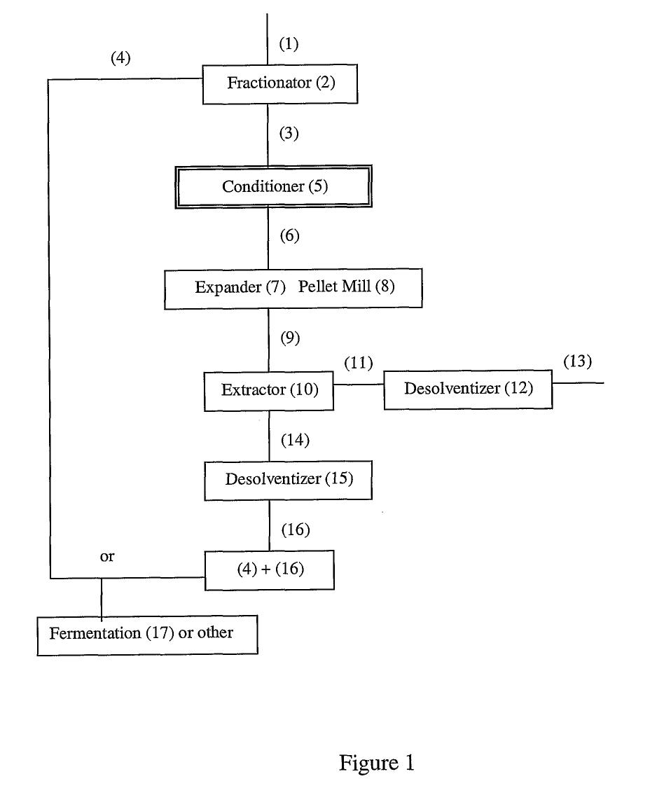

Referring to FIG. 1, in one embodiment of the process of the present invention, incoming whole corn kernels (1) are conveyed into a fractionator (2). Suitable fractionating equipment includes the Buhler-L apparatus (Buhler GmbH, Germany), a Satake VCW debranning machine (Satake USA, Houston, Tex.), or other apparatus wherein the incoming corn material is contacted with an abrasive device such as a screen to remove the hull and the germ component of the corn material from a portion of the remainder of the corn material (endosperm component). As used herein, “germ component” refers to a portion of the corn material containing corn germ, fractions of corn germ, components of germ, or oil-bodies. Some of the germ component and hull are removed from the corn kernels by pushing and rubbing the kernels at and against the screen. The removed germ component and the bran go through the screen and form a higher oil fraction (“HOF”) (3). The material left on the screens (the endosperm component) is a lower oil fraction (“LOF”) (4), but will contain some germ component. The HOF has an oil concentration greater than that of the corn kernels and the LOF has an oil concentration less than that of the corn kernels. As used herein, “endosperm component” refers to a portion of the corn material containing endosperm, or components of endosperm (starch and protein from outside the germ). The HOF is predominantly less than size US #18 mesh sieve having a 1.00 mm opening as defined in ASTME-11 specifications. Separation of HOF is enhanced by maintaining a negative pressure on the outer screen cage of the Buhler L.

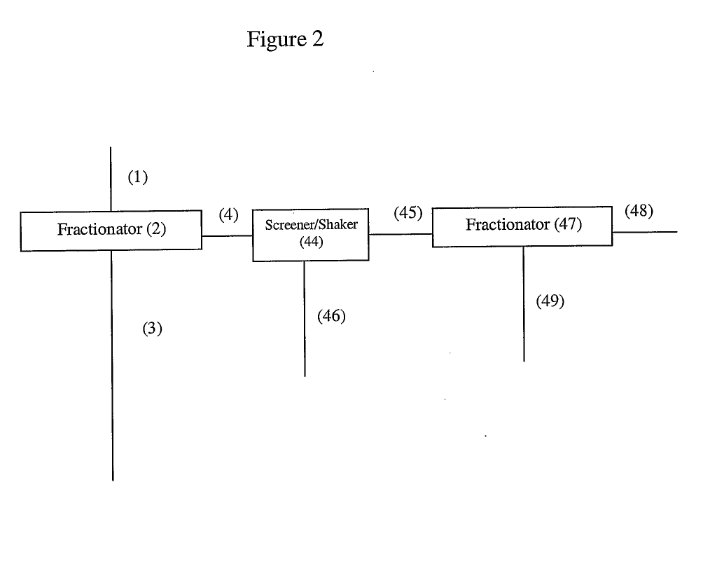

A preferred embodiment includes a second fractionation step. Referring to FIG. 2, incoming whole corn kernels (1) are conveyed into a fractionator (2). The resulting LOF (4) is screened using a vibrating screening and shaking device (44), such as that manufactured by Rotex (Rotex, Inc., Cincinnati, Ohio, Model #201GP) or Buhler the “MPAD Pansifter”). A 6000 micron screen is preferred. Particles of the LOF size A5-30 and larger (45) are then conveyed to a second stage fractionator (47), which in one embodiment is a duplicate of the first stage fractionater. The resulting second-stage LOF stream (48) can be combined with the stream of screened particles of the LOF less than size A5-30 (46) to form combined LOF (51). Similarly, the second-stage HOF stream (49) can be combined with the HOF stream (3) to form combined HOF (50).

In the following paragraphs, unless otherwise specified, where the second stage fractionation step is used, HOF (3) may be replaced in whole or part by combined HOF (50) or second stage HOF stream (49). The HOF (3) may be conditioned. Conditioning may be used when the residual oil level of the HOF exceeds about 6%. The HOF may be conditioned (5) using methods known to those of ordinary skill in the art. As used herein, the term “conditioning” refers to a process by which the corn material is heated prior to expansion to improve the plasticity of the expandette. As used herein, an “expandette” is what is produced by submitting the HOF (3) or conditioned HOP (6) to an expander. An expandette is also referred to herein as “expanded, shaped HOF”. As used herein, “plasticity” refers to the combination of properties of an expandette: how well it holds together in a structure, that it contains a low amount of fines, it has a high level of structural integrity, it has high porosity (good drainage), and it has low complexation between oil and starch. As used here, “fines” refers to particles that pass through a US #18 mesh sieve having a 1.00 mm opening as defined in ASTME-11 specifications. The amount of fines is determined by sifting. The amount of fines should be less than about 20 wt. % and preferably is less than about 10 wt. %. The porosity, complexation, and extractability can be determined as described in Aguilera et al., “Laboratory and Pilot Solvent Extraction of Extruded High-Oil Corn”, JAOCS, 63(2):239-243 (1986). Structural integrity may be determined by testing in a Model 2 Crown pilot extractor. Acceptable results are that the recirculation pump does not plug, drainage is acceptable to an experienced operator and the residual oil in the meal is less than about 2.0 wt. %. A preferred residual oil level in the meal is less than about 1.5 wt. %. Optimum quality of expandettes are produced with a moisture level of between about 10% and about 14%.

Conditioning may include the addition of steam (saturated and/or superheated) and/or water to the corn. The conditioning temperatures range between about 25° C. and about 95° C. and the moisture can be increased up to about an additional 10% of the moisture. It is preferred to use corn material which does not need to be conditioned. One conditioner which may be used is a Buhler homogenizer (Model DPSD, Buhler Gmbh, Germany). The HOF (3) or conditioned HOF (6) is conveyed to an expander (7) and/or pellet mill (8). The expanded and/or pelleted HOF (9) is subjected to extraction (10) to recover the extracted oil (11) and the extracted meal (14). It is preferred to expand the HOF rather than to pellet the HOF, as a pellet is generally not as extractable as an expandette.

A useful expander (7) is the Buhler Condex Expander DFEA-220 (Buhler GmbH, Germany) fitted with a 30 slot 8 mm die head to form shaped expanded HOF (9). The shaped, expanded HOF (9) is cut to desired lengths by the expander. Steam is sparged into the expander barrel to give the corn material the plasticity to produce the shaped, expanded HOF (9). Other expanders can be used. Those of skill in the art are familiar with adjusting the operating conditions of an expander in order to provide the requisite plasticity to the expandette. Using an expander or pellet mill provides the HOF in a solvent-extractable structure without using flaking.

Corn oil (11) is extracted from the HOF (9) by one or more extraction steps using any extraction method. Generally, substantially, or about all of the oil is extracted in a single extraction process. At least about 5, 10, 15, 20, 25, 30, 35, 40, 45, 50, 55, 60, 65, 70, 75, 80, 85, 90, or 95% of the oil is extracted. Useful extraction methods include solvent extraction, continuous solvent extraction, hydraulic pressing, expeller pressing, aqueous and/or enzyme extraction. Useful solvents for solvent extraction include, for example, hydrocarbons, alkanols, alkanol-containing aqueous solutions, and supercritical carbon dioxide. Examples of such solvents include, but are not limited to C₂-C₈ hydrocarbons, C₁-C₄ alkanols, including methanol, ethanol and isopropanol. Mixtures of solvents may be used. Hexane(s) is a preferred solvent. For example, corn oil can be extracted from the HOF (9) using a hexane-based solvent extractor. Solvent extractors can include both percolation and immersion type extractors. In a preferred embodiment, a continuous solvent extraction process allows the HOF (9) to remain in contact with the solvent for at least 10 minutes, at least 30 minutes, or at least 45 minutes. The less time the HOF is in contact with the solvent, the more economical the extraction process. Equipment used for the extraction of oil from oilseeds, such as soybean and canola, can be used to prepare the extracted corn oil and extracted corn meal described herein.

Materials removed from solvent-based extractors include extracted corn meal (14) and extracted corn oil in the form of miscella (11). A miscella is a mixture comprising extracted oil and solvent. The extracted corn meal comprises the materials that remain after some or all of the solvent-soluble material has been extracted. The extracted corn meal (14) also contains a quantity of solvent. Solvent is reclaimed from both the miscella and the extracted corn meal using methods such as rising film evaporation, or drying, and raising the temperature using equipment such as flash tanks and/or de-solventizer/toasters (12, 15). For example, heat is applied to the extracted corn meal or miscella under atmospheric pressure, under elevated pressure, or under vacuum to evaporate the solvent. The evaporated solvent is then condensed in a separate recovery system, and optionally dewatered and recycled to the extractor. Alternatively, in case of using an alkanol or its solution as an extractant, oil separation from the miscella could be done by adding water to the miscella. Such addition results in division of the miscella into two phases. The first is an oil phase containing only small amounts of alkanol, which could be removed by distillation. The other is an aqueous solution of the alkanol, which, if required, could be re-concentrated. In a plant containing an ethanol production operation, if ethanol is used as oil extractant, re-concentration could be combined with ethanol separation from the fermentation liquor.

Desolventized miscella (13) is commonly termed crude oil, which can be stored and/or undergo further processing. Crude oil can be refined to produce a final oil product. Methods for refining crude oil to obtain a final oil are known to those of ordinary skill in the art. Hui (1996) provides a thorough review of oils and oilseeds (Bailey's Industrial Oil and Fat Products, Fifth Ed., Vol. 2, Wiley and Sons, Inc., New York, 1996). Chapter three of Hui (pp. 125-158), the disclosure of which is hereby incorporated by reference, specifically describes corn oil composition and processing methods. Crude oil isolated using the methods described herein is of a high quality but can be further purified as needed using conventional oil refining methods. The refining may include bleaching and/or deodorizing the oil or mixing the oil with a caustic solution for a sufficient period of time to form a mixture and centrifuging the mixture to separate the oil.

The LOF, which contains endosperm component, is a stream higher in starch as compared to yellow #2 corn grain. This stream can be utilized for many applications in the food, chemicals, and industrial products industries. Due to its high starch content and lower oil and fiber concentrations as compared to yellow #2 corn, this stream is an ideal feed source for many fermentation processes, including, but not limited to ethanol and butanol production. Other uses include using this as feedstock material to produce carboxylic acids, amino acids, proteins, and plastics as well as cosmetics and food applications.

In the following paragraphs, one of skill in the art will appreciate that, unless otherwise specified, where the second stage fractionation step is used, LOF (4) may be replaced in whole or in part by combined LOF (51) or second stage LOF stream (48), respectively.

In one embodiment, the HOF (3) is combined with other oil-containing corn material to be extracted (e.g., corn germ) and formed into a solvent extractable structure prior to extraction. In one embodiment, expanded HOF (9) is combined with other oil-containing corn material already in a solvent-extractable structure as a feedstock for extraction.

In one embodiment, the LOF (4) is used as a feedstock for fermentation, corn wet milling, pet food, animal feed, food applications, and/or other processes. In another embodiment, the LOF is combined with the extracted, desolventized meal (16) and used as a feedstock for fermentation, corn wet milling, pet food, animal feed, food applications, and/or other processes. In one aspect of the present invention, the extracted, desolventized meal (16) can be used as a feedstock for fermentation, corn wet milling, pet food, animal feed, food applications, and/or other processes.

Analysis of components of exemplary LOF ((4) only) samples provided the following:

| | Run 1 | Run 2 | Ave | DB | CV |
|---|---|---|---|---|---|
| Ash, % | 0.62 | 0.62 | 0.62 | 0.75 | 0.0% |
| Moisture, % | 17.71 | 17.70 | 17.71 | 0.00 | 0.0% |
| Fat, % | 1.34 | 1.42 | 1.38 | 1.68 | 4.1% |
| Protein, % | 6.31 | 6.27 | 6.29 | 7.64 | 0.4% |
| Acid | 2.20 | 2.10 | 2.15 | 2.61 | 3.3% |
| detergent | | | | | |
| fiber “ADF”, % | | | | | |
| Neutral | 5.60 | 5.20 | 5.40 | 6.56 | 5.2% |
| detergent | | | | | |
| fiber “NDF”, % | | | | | |
| Starch, % | 68.42 | 68.79 | 68.61 | 83.36 | |

DB = dry basis

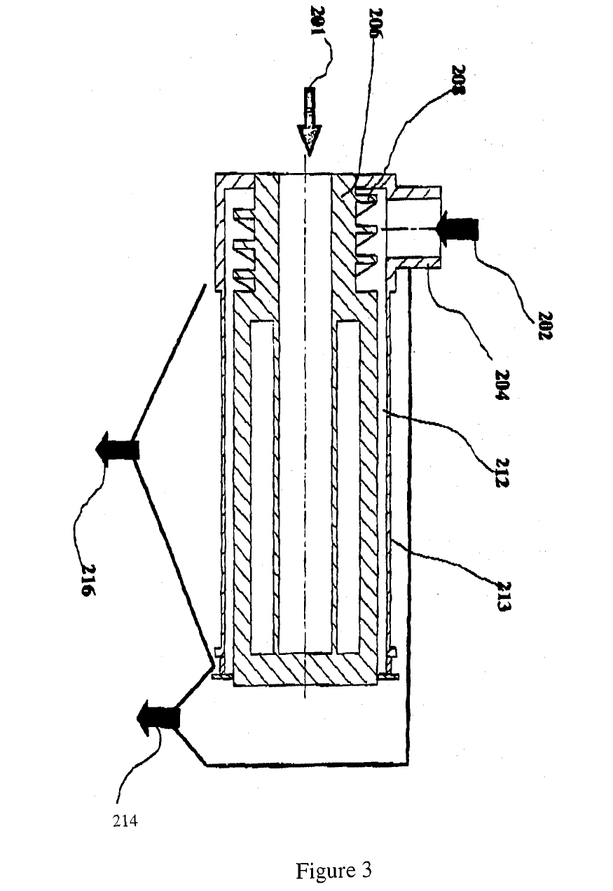

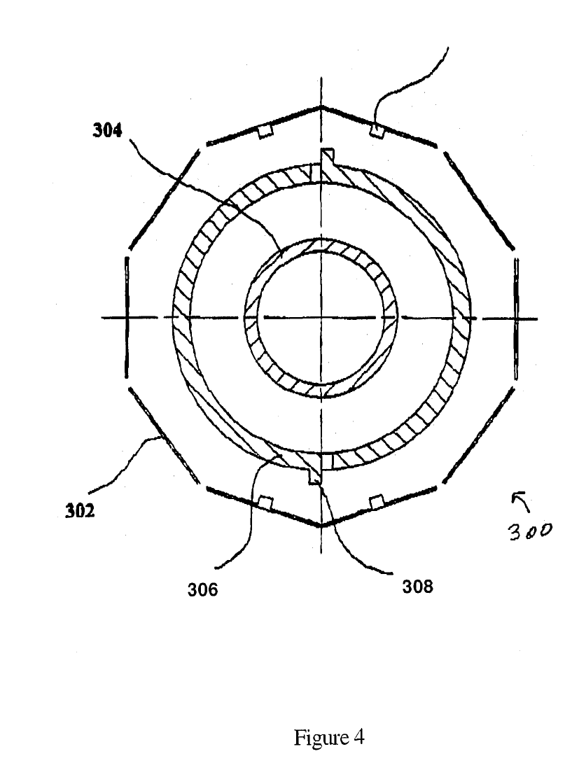

FIG. 3 is a longitudinal section view of the fractionating apparatus (2) shown in FIG. 1. In one embodiment corn material (e.g. corn kernels) (202) are conveyed into the apparatus through a cylindrical intake pipe (204) which moves the corn material into a horizontal tunnel which has rotating screw (206) going through the tunnel. The rotating screw has longitudinal bars (as seen in cross section in FIG. 4 at 308) running its length and spiral flights (208) to convey the corn material into the cylindrical mill (212) which has flat polygonal sides. Air (201) pushes down through into the horizontal cylindrical mill. The corn material is pushed down the tunnel by the flights and are rubbed against the flat polygonal screens which form the sides of the cylindrical mill (212). The action of the corn material against these screens abrasively removes the HOF (3), which goes through the screens and exits the mill at conduit (216) where a pressure plate (not shown) is resiliently mounted, such as with springs, over the exit of the mill to cover the exit of the mill and in part control the pressure being exerted on the corn material being pushed against the slits (see 307 in FIG. 4) of the mill. The LOF (4) stays within the cylindrical mill (212) and is then conveyed by the screw down the exit (214).

FIG. 4 shows a cross section view of the screen-sided cylindrical mill (212). The polygonal-sided cylindrical mill (300) has flat sides (302) which are screens. Rotating or turning rollers (306) are rotated by axle (304). Nips (308) revolve within the screen and rub the corn material against the screen to remove the HOF (3).

In one embodiment, the screens which form polygonal sides of a cylinder have rectangular holes or slits (307) (as opposed to round holes) having dimensions of 1 to 3 mm by 20 to 25 mm. The corn material is pushed outwardly from the inside of the polygonal sided cylindrical mill (212) with the corn material being pushed by cylindrical-shaped rotating rotors (306) inside the cylindrical shaped mill. The mill does not have a reduced diameter moving from the inlet to the outlet of the mill. The cylindrical mill (212) with slits (307) is stationary with the corn material being impelled horizontally down the length of the cylinder and outwardly from the longitudinal axis of the cylinder by the rotating cylindrical rotors to the slitted or slotted polygonal sides of the cylinder. The fractionating is done preferably with a Buhler-L apparatus which has six flat polygonal sides with rectangular slits and a cylindrical-shaped rotor. See, e.g., WO 04/041434.

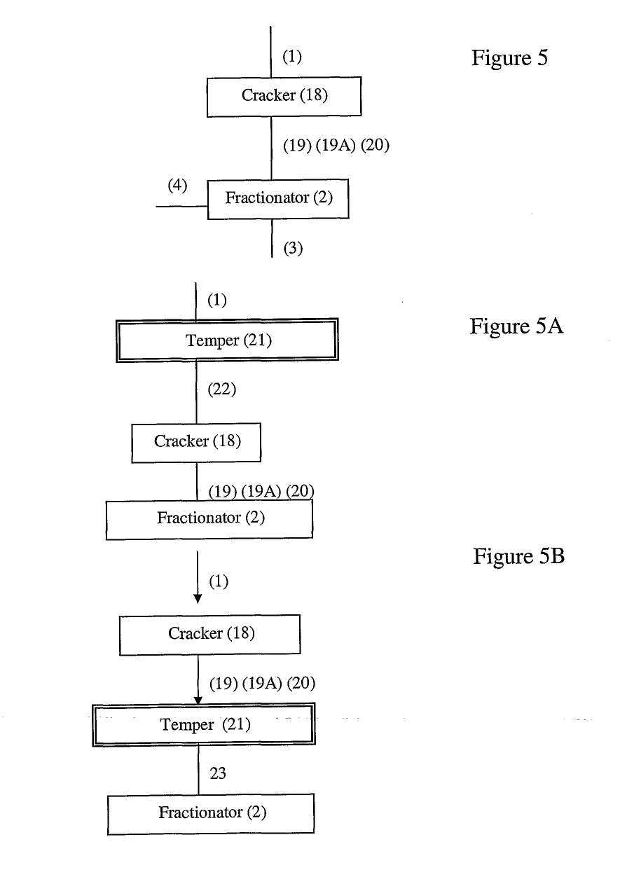

FIG. 5 depicts an alternative embodiment of the present invention. Corn kernels (1) are conveyed to a cracking apparatus (18) prior to entering the fractionating apparatus (2). The corn kernels may be cracked by passing them between two rollers with corrugated teeth spinning toward each other spaced by a defined gap, and/or passing through a grind mill where a rotating toothed disk spins at an adjustable distance from a stationary disk. Methods for cracking corn or high oil seeds are described in Watson, S. A. & P. E. Ramstad, ed. (1987, Corn: Chemistry and Technology, Chapter 11, American Association of Cereal Chemist, Inc., St. Paul, Minn.), the disclosure of which is hereby incorporated by reference in its entirety. A “cracked” corn is a corn that has undergone the above-described cracking process.

A preferred cracking apparatus is the Roskamp series 900 cracking mill roller (Roskamp, Waterloo, Iowa) with corrugated rollers with 6 teeth per inch in a round bottom v design. Other cuts can also be used, including, but not limited to, a modified Dawson cut, or a LePage cut. The roller gaps of the rollers are adjusted based upon on the quality of the incoming grain.

The corn kernels are cracked into at least two sizes of cracked corn pieces. Preferably, there are three sizes of cracked corn pieces: the large size pieces of cracked corn (19), the medium size pieces of cracked corn (19A) and the small size pieces of cracked corn (20). Preferably, the small size pieces of the cracked corn comprise less than about 10 wt. % of the cracked corn pieces. The small size pieces of the cracked corn are less than about 1080 microns in size. Preferably, the medium size pieces of cracked corn (19A) comprise about 70 wt. % of the cracked corn. Preferably, the medium size pieces comprise predominantly endosperm component. As used herein, “predominantly” refers to about 90% or greater. Preferably, the medium size pieces range in size from about 2540 microns to about 4270 microns. The large size pieces of cracked corn comprise about 20 wt. % of the cracked corn. Preferably, the large size pieces of cracked corn (19) comprise about 30 wt. % to 40 wt. % germ component. Preferably, the large size pieces of the cracked corn are greater than about US #4 mesh screen in size (4750 microns)

In one embodiment, the large, medium, and small size pieces of cracked corn are fed into the fractionating apparatus (2) and are subjected to the remainder of the process described above in connection with FIGS. 1, 3, and 4.

In any of the embodiments described above, and as depicted in FIGS. 5A and 5B, the corn kernels or the pieces of cracked corn are optionally tempered (21) prior to either the cracking or fractionating steps. Tempering refers to heating the corn material directly or indirectly and/or adding moisture to the corn material. Tempering is a means to uniformly distribute the added moisture and/or heat through the corn material. The tempering adds up a maximum of about 1%, 2%, or 3% additional moisture to the corn material. The tempering is done to increase the differential hardness between the germ component and the remainder of the corn material. A preferred method of tempering is heating the corn indirectly.

Any tempering method known in the art is acceptable, including, but not limited to spraying water or sparging steam. A preferred method of tempering is to use a stacked cooker or rotary steamed tube heater. Alternatively, a steam jacket mixer may be used. In general, the corn is tempered in an appropriate amount of water for any suitable length of time, such as at least about 15 seconds, 30 seconds, 45 seconds, 1 minute, and increasing in about 15 second increments up to about at least 30 minutes. A preferred time for tempering, if tempering is used, is about 2 minutes.

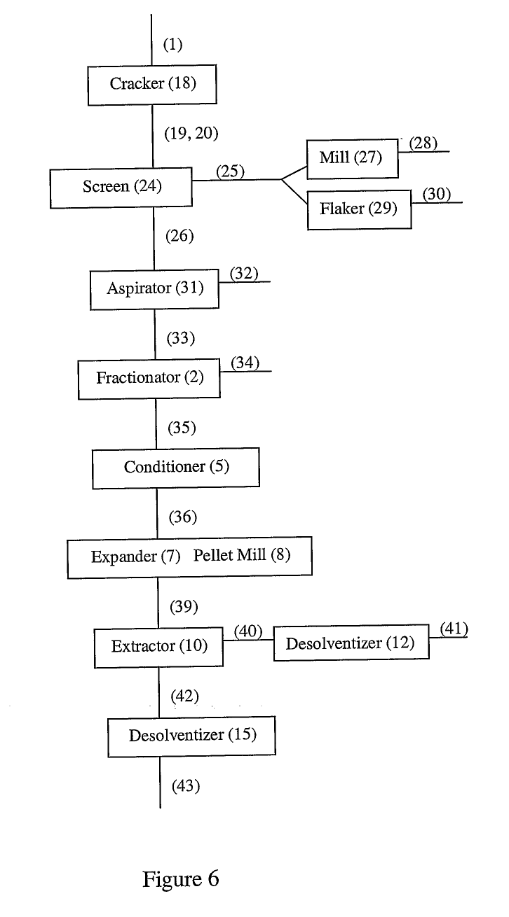

An alternative embodiment of the present invention is depicted in FIG. 6. Corn kernels (1) are conveyed into a cracking apparatus (18) as described above. After cracking, the large size pieces of cracked corn (19) and medium size pieces of cracked corn (19A) are separated from the small size pieces of cracked corn (20), such as by screening (24). One screen which can be used in the process of the present invention is a Rotex screen with a 4 mesh mill grade with 5.46 mm holes (Rotex, Inc., Cincinnati, Ohio, Model #201GP). Other methods of separation include, but are not limited to, other methods of size separation or gravity separation known to those skilled in the art, such as, but not limited to, aspiration and cyclonic separation.

The medium and large size pieces of cracked corn (25) are retained by the screen (24). The retained medium and large size pieces of cracked corn are ground in a mill (27) or flaked in a flaker (29). A useful mill (27) is the Fitzmill comminuter (Fitzpatrick Company, Elmhurst, Ill.) fitted with a ¼ inch screen. Useful commercial-scale oilseed flakers (29) can be obtained from French Oil Mill Machinery Company, Piqua, Ohio; Roskamp Champion, Waterloo, Iowa; Buhler AG, Germany; Bauermeister, Inc., Memphis Term.; Consolidated Process Machinery Roskamp Company, on the world wide web at http://www.cpmroskamp.com, and Crown Iron Works, Minneapolis, Minn.

For high oil corn, preferably, the large size pieces of cracked corn (19) comprise from about 11 wt. % to about 22 wt. % oil. For high oil corn, preferably the medium and small size pieces of cracked corn (19A, 20) comprise from about 4.5 wt. % to about 8 wt. % oil.

After being ground in the mill, the ground cracked corn (28) is added to the stream being fed to the expander (7) or pellet mill (8). After being flaked, the flaked cracked corn (30) is added to the stream exiting the expander (7) or pellet mill (8).

Optionally, the small size screened pieces of cracked corn (26) may be aspirated (31) to remove fines (bran) (32). In one embodiment, the bran is added to the feed to the extractor. In one embodiment, the bran is extracted separately from other corn components. In one embodiment, the bran is used as a feedstock from which to extract one or more components of the bran, e.g. phytosterols. In one embodiment, the bran is used as fermentation feedstock. In yet another embodiment, the bran is used in a cattle feed. In an alternative embodiment, the aspiration of the bran is not performed until after the fractionation step. In this embodiment, the HOF (3) is aspirated to remove the bran.

In either aspiration embodiment, a separate bran (fiber) stream results. This stream contains elevated levels of pericarp carbohydrates as compared to yellow #2 corn. The sugars associated with the fiber or pericarp are typically pentoses, which are 5 carbon sugars such as Arabinose and Xylose. These carbohydrates have many uses in the food, industrial chemicals, and fuels markets. Because this stream has an elevated concentration of these key sugars, this stream can be used as a feedstock material to separate the carbohydrate sugars of interest. In addition, this stream can be feed directly into an ethanol fermentation process to utilize the sugars to produce ethanol. The bran (fiber) stream also contains valuable components such as phytosterols.

The HOF without the bran is a higher oil fraction without (or with less) fiber stream. This stream contains elevated oil and protein concentrations compared to yellow #2 corn grain and is a potential feed source for industrial applications and has unique food uses. Due to its higher protein levels, this stream can be a good feed source for water, aqueous solution, salt, pH, membrane, and/or alcohol protein extraction. This can lead to a protein concentrate for use in the food and industrial chemicals industry. Additionally this stream can be further processed via extraction with a solvent and/or the use of water and ultrasound for protein(s), amino acids, oil or novel compounds such as nutraceuticals and carotenoids. Ethanol or an ethanol solution is a suitable extractant, according to an exemplary embodiment. According to a preferred embodiment, the ethanol is produced by fermenting a kernel fraction. Extracted components could be separated from the extract formed in the extraction, e.g. by distillation of the solvent. Such distillation is combined with distillation in an ethanol production part of the plant. The HOF with the bran can be used as an animal feed source or as a food additive.

The small size screened pieces of cracked corn (26) and/or screened and aspirated small size pieces of cracked corn (33) are fed to the fractionator (2), which separates them into a higher oil cracked fraction (35) and a lower oil cracked fraction (34). In one embodiment, the lower oil cracked fraction is used as a feedstock for fermentation, corn wet milling, pet food, animal feed, food applications, and/or other processes. In one embodiment, the lower oil cracked fraction is combined with extracted corn meal (16). The combination may be used as a feedstock for fermentation, corn wet milling, pet food, animal feed, food applications, and/or other processes.

The higher oil cracked fraction (35) is optionally conditioned (5) and then conveyed to the expander (7) or pellet mill (8). Oil is then extracted (10) from the expanded cracked higher oil fraction (39), alone or in combination with the flaked cracked corn (30) and/or the ground cracked corn (28).

What remains after extraction of the expanded HOF (9), the expanded, cracked HOF (39), the flaked cracked corn (30), and the ground cracked corn (28) is an extracted corn meal—a protein stream higher than yellow #2 corn. Because this stream contains very little oil, it can be used to produce protein concentrates as well as protein isolates via an alcohol and/or water extraction process. Additional uses for this stream include production of plastic pre-cursors from the proteins associated with this stream for amino acids or novel compound separation as well as other uses described herein.

Analysis of components of exemplary extracted HOF samples from high oil corn provided the following:

| | Run 1 | Run 2 | Ave | CV |
|---|---|---|---|---|
| Ash, % | 2.66 | 2.68 | 2.67 | 0.5% |
| Moisture, % | 8.71 | 8.66 | 8.69 | 0.4% |
| Fat, % | 2.18 | 2.23 | 2.21 | 1.6% |
| Protein, % | 11.35 | 11.29 | 11.32 | 0.4% |
| ADF, % | 4.30 | 4.50 | 4.40 | 3.2% |
| NDF, % | 14.60 | 14.90 | 14.75 | 1.4% |

In one embodiment, one or more of the HOF (3), expanded HOF (9), the higher oil cracked fraction (35), the expanded/pelleted higher oil cracked fraction (39), as such, or after extraction, are used as fermentation feedstock.

U.S. Pat. No. 6,313,328 describes commercial-scale methods and equipment as sufficient for extracting corn oil from at least about 1 ton of corn per day. In some embodiments, the capacity of commercial-scale operations ranges from about 100 tons of corn per day to about 3000 tons of corn per day, or the capacity ranges from about 700 tons of corn per day to about 1700 tons of corn per day. Commercial-scale operations that process greater than about 3000 tons of corn per day are also sufficient. In contrast, the process of the present invention allows for processing of up to 10,000 tons daily.

The extracted corn oil and/or extracted corn meal and/or LOF of the present invention may be combined with a variety of other ingredients. The specific ingredients included in a product will be determined according to the ultimate use of the product. Exemplary products include animal feed, raw material for chemical modification, biodegradable plastic, blended food product, edible oil, cooking oil, lubricant, biodiesel, snack food, cosmetics, and fermentation process raw material. Products incorporating the meal described herein also include complete or partially complete swine, poultry, and cattle feeds, pet foods, and human food products such as extruded snack foods, breads, as a food binding agent, aquaculture feeds, fermentable mixtures, food supplements, sport drinks, nutritional food bars, multi-vitamin supplements, diet drinks, and cereal foods.

When making oil-based products according to the present invention, those products can include conventional corn oil, soy oil, canola oil, olive oil, palm oil, sunflower oil, safflower oil, antioxidant, flavoring, hydrogenated oil, partially hydrogenated oil, and/or animal fat. By mixing the corn oil herein with one or more other oils, blended oil products are made. The corn oil-based products can also include materials such as food additives, salt, fat, food colors, β-carotene, annatto extract, curcumin or tumeric, β-apo-8′-carotenal and methyl and ethyl esters thereof, natural or synthetic flavors, antioxidants, propyl gallate, butylated hydroxytoluene, butylated hydroxyanisole, natural or synthetic tocopherols, ascorbyl palmitate, ascorbyl stearate, dilauryl thiodipropionate, antioxidant synergists, citric acid, sodium citrate, isopropyl citrate, phosphoric acid, monoglyceride citrate, anti-foaming agent, dimethyl polysiloxane, crystallization inhibitor, oxystearin, amino acids, vitamin, minerals, carbohydrates, sugars, herbs, spices, acidity regulators, firming agents, enzyme preparations, flour treatment agents, viscosity control agents, enzymes, lipids, and/or vegetable or animal protein. Additionally, these edible products can be enhanced or enriched with protein supplements containing utilizable protein. An exemplary food product such as a breakfast cereal could include ingredients such as meal of the present invention, wheat and oat flour, sugar, salt, corn syrup, milled corn, dried fruit, vitamin C, B vitamins, folic acid, baking soda, and flavorings. Other exemplary oil-based products that can comprise the oil prepared herein include food oil, cooking oil, edible oil and blended oil.

The crude oil prepared according to the methods described herein can be subsequently partially or completely hydrogenated. Suitable methods for partially or completely hydrogenating oil are described in D. R. Erickson, Practical Handbook of Soybean Processing Utilization (1995, AOCS Press), the entire disclosure of which is hereby incorporated by reference.

The extracted corn oil can be used as a raw material for chemical modification, a component of biodegradable plastic, a component of a blended food product, a component of an edible oil or cooking oil, lubricant or a component thereof, biodiesel or a component thereof, a component of a snack food, a fermentation process raw material, or a component of cosmetics. When making blended oils with the extracted oil, the blending can be done before, during, or after the extraction process.

Biodiesel can be produced using the extracted corn oil of the present invention. Biodiesel is a general term used for a variety of ester-based oxygenated fuels. Biodiesel produced today is a mixture of fatty acid methyl esters produced by methylating refined vegetable oil. Refined oil is preferable to crude oil or spent fryer oil due primarily to the quality of the glycerol by-product. The main drawbacks with previous biodiesel products and related vegetable oil lubricants are low temperature properties and reactivity toward oxidation and polymerization. A preferred biodiesel product comprises a low cloud point, reduced stearic and polyunsaturated fatty acid content, and high oleic acid content. Pour point correlates with low temperature properties and is influenced by the saturated fatty acid content of the oil. Polyunsaturated fatty acids are more susceptible to oxidation and polymerization reactions.

Extracted corn oil (“ECO”) produced by the process of the present invention exhibits improved cloud point performance over soy, while exhibiting similar chemical stability.

Extracted corn oil produced by the present invention can be further processed to form lubricants such as by published procedures practiced currently in the industry (see, e.g., U.S. Pat. No. 6,174,501).

One aspect of the present invention provides a nutritious animal feed comprising the extracted corn meal and/or LOF produced by the present invention. The animal feed can comprise other nutritious products such as vitamins, minerals, high oil seed-derived meal, meat and bone meal, salt, amino acids, feather meal, fat, oil-seed meal, corn, sorghum, wheat by-product, wheat-milled by-product, barley, tapioca, corn gluten meal, corn gluten feed, bakery by-products, full fat rice bran, rice hulls, and many others used in the art of feed supplementation. The animal feed composition can be tailored for particular uses such as for poultry feed, poultry layer feed, swine feed, cattle feed, equine feed, aquaculture feed, pet food, and can be tailored to animal growth phases. Particular embodiments of the animal feed include growing broiler feed, swine finishing feed, and poultry layer finishing feed. Feed products can be made with the extracted corn meal that will have a higher relative percentage of protein and lower relative percentage of oil than similar products made with conventional corn.

Another aspect of the present invention provides a corn oil-based product comprising corn oil obtained by extraction of at least some of the endosperm component and some of the germ component of high oil corn. The corn oil-based product can comprise other components such as vinegar, spices, vitamins, salt, hydrogen (for forming hydrogenated products), and water. The corn oil used in the products of the present invention will generally contain a higher proportion of β-carotene, xanthophylls, or tocotrienol than similar products made with corn oil extracted from conventional corn employing conventional methods. This corn oil, from the process of the present invention, is generally produced by exposing both the endosperm component and the germ component to extraction. Therefore, the solvent-extractable nutrients present from the endosperm component are extracted into the corn oil that has been extracted from both endosperm and germ components. Products that can be made with such oil include, but are not limited to, salad dressings, cooking oils, margarines, spray-coated food or feed products, breads, crackers, snack foods, lubricants, and fuels.

Another aspect of the present invention provides a method of using extracted corn meal and/or a lower oil fraction in an animal feed ration comprising the step of: 1) providing an extracted corn meal and/or lower oil fraction prepared by the present invention; and 2) including the extracted corn meal and/or lower oil fraction in an animal feed ration. It will be appreciated by those skilled in the art that the preferred ratio of extracted corn meal and LOF in a product will approximate the amount of each in the corn kernel, less the oil.

Another aspect of the present invention provides a method of using an extracted corn oil in a food product comprising the steps of: 1) providing an extracted corn oil obtained by a method of the present invention; and 2) including the extracted corn oil in a food product.

Another aspect of the present invention provides a method of using extracted corn oil as a feedstock in an oil refining process. The method comprises the steps of: 1) providing an extracted crude corn oil obtained by a method of the present invention; and 2) including the extracted crude corn oil in a raw material stream of an oil refining process.

Another aspect of the present invention provides a method of using extracted corn oil from a process of the present invention as an ingredient in cosmetic applications. The method comprises the steps of: 1) providing an extracted crude corn oil obtained by a process of the present invention; and 2) including the extracted crude corn oil in a cosmetic product. These types of cosmetics include but are not limited to lipstick and eye liner. Another aspect of the present invention provides the use of an extracted corn meal and/or lower oil fraction in an animal feed or human food, wherein the extracted corn meal is obtained by a process of the present invention. Yet another aspect of the present invention provides the use of a corn oil in an animal feed or human food, wherein the corn oil is obtained by a process of the present invention.

Corn oil or corn meal quality is determined by evaluating one or more quality parameters such as the oil yield, phosphorus content, free fatty acid percentage, the neutral starch percentage, protein content, and moisture content. Any method can be used to calculate one or more of the quality parameters for evaluating the oil or meal quality.

The lower oil fraction (4) and the extracted corn meal (16) can be provided as a loose product or a pelleted product, optionally in combination with other components. For example, a pelleted product could include the extracted corn meal (by itself or in combination with other components) that has been pelleted and subsequently coated with zein protein. The corn meal can be included in blended meal products which can be provided in loose or pelleted form. Meal produced from the processes described herein is used to produce feed products. Blended meals may comprise the following ingredients in the approximate amounts: 0.5-12% fat, 5-45% moisture, 5-60% protein, 2-4% crude fiber, and 40-80% carbohydrates.

Feed products containing predominantly corn meal produced by extraction require less supplementation with protein from other sources such as soybeans than feed products containing predominantly normal corn grain. The meal, by virtue of the composition arising from the processing method, offers feed manufacturers flexibility to produce feeds that could otherwise not be made. Animal feed rations having unique physical properties such as bulk density, texture, pelletability, and moisture holding capacity and/or unique nutritional properties are created by including the extracted corn meal of the present invention as a component of said rations. The extracted corn meal isolated using methods as described herein can, on its own, be a low-fat corn meal. Alternatively, it can be used in combination with the lower oil fraction produced by the present invention, and/or other corn meals or nutritional components to make feed rations and food products. The extracted corn meal and/or lower oil fraction can also be combined with meals made from crops such as soybeans, canola, sunflower, oilseed rape, cotton, and other crops. The extracted corn meal and/or lower oil fraction can also be made from genetically modified corn and/or combined with meals made from transgenic oilseed grains to form an enhanced meal or enhanced product.

The feed rations prepared with the extracted corn meal and/or lower oil fraction will generally meet the dietary and quality standards set forth in the CODEX ALIMENTARIUS or by the National Research Council. The corn meal of the present invention will generally comprise the components in the approximate amounts indicated in Table 2 below.

### Table 2

TABLE 2

| | Sample A | Sample B | Sample C |
|---|---|---|---|
| Component | Amount (%) | Amount (%) | Amount (%) |
| Moisture | 5-45 | 5-25 | 5-45 |
| Starch | 40-70 | 40-80 | 40-70 |
| Protein | 8-20 | 7-20 | 8-20 |
| Fat (Oil) | 0.75-6 | 0.75-6.0 | 0.75-12 |
| Crude Fiber | 2-4 | 2-4 | |
| Ash | 1.5-3 | 0.5-2.0 | |
| Fructose | 0.15-0.3 | | |
| Glucose | 0.2-0.5 | | |
| Sucrose | 1.5-2.5 | | |
| Lysine | | | 0.15-2.0 |
| Tryptophan | | | 0.03-2.0 |

The corn meals above may also further comprise unspecified amounts of the components for which no amounts have been indicated. In one embodiment, the extracted corn meal comprises the components in the approximate amounts: about 0.5 to 12 wt. % fat, 5-45% moisture, 7-20% protein, 4-11% crude fiber, and 40-80% carbohydrates.

The lower oil fraction will generally comprise these components in these approximate amounts: moisture 5-25%, oil 1-3.5%, protein 9-12%, starch 40-80%, fiber 2-6%, and ash 0.5 to 2%. The lower oil fraction may also further comprise other components.

Varying levels of nutrients are required by different animals depending on species, age, and breed. Feed rations comprising different levels of nutrients are made by subjecting the high oil corn to different degrees of extraction, i.e., more oil is removed from the corn by subjecting it to extraction to a greater degree. Therefore, feed rations comprising the extracted corn meal of the present invention can be made to include different amounts of fat, protein, and carbohydrates by controlling the extent to which the high oil corn is extracted. Table 3 details the amounts in which the indicated ingredients are present in animal feed rations comprising the extracted corn meal, the specific inclusion range being indicative of exemplary rations in which extracted corn meal is a main ingredient and the general inclusion range being indicative of rations in which one or more other ingredients, for example, carbohydrate-based energy sources such as sorghum, wheat, and/or other cereal grains or their by-products, or other non-cereal grain ingredients, may be included.

### Table 3

TABLE 3

| | General | Exemplary |
|---|---|---|
| Ingredient | Inclusion Range | Inclusion Range |
| Corn meal described herein | 2-95% | 50-90% |
| Oilseed Meal¹ | 3-35% | 10-30% |
| Meat and Bone Meal | 0-12% | 0-7% |
| Feather Meal | 0-6% | 0-4% |
| Fat | 0-10% | 1-6% |
| Salt | 0.1-0.5% | 0.1-0.5% |
| Lysine | 0-0.4% | 0-0.4% |
| Methionine | 0-0.3% | 0-0.3% |
| Nutrient Premix | 0.01-1.0% | 0.01-1.0% |

¹Oilseed meal can consist of, but is not limited to, soy, sunflower, canola, cottonseed, and other plant-based meals, which themselves may or may not have been subjected to an oil extraction process.

Meat and bone meal is obtained from suppliers such as Darling International, Inc. (Irving, Tex.). Oilseed meal is obtained from suppliers such as Cargill Oilseeds (Cedar Rapids, Iowa). Feather meal is obtained from suppliers such as Agri Trading Corp., (Hetchinson, Minn.). Amino acids are obtained from suppliers such as DuCoa, (Highland, Ill.).

Feed rations are made by mixing various materials such as grains, seed meals, vitamins, and/or purified amino acids together to form a composite material that meets dietary requirements for protein, energy, fat, vitamins, minerals, and other nutrients. The mixing process can include grinding and blending the components to produce a relatively homogeneous mixture of nutrients. Physical properties of the feed raw materials and of the compounded feed affect the nutritional quality, storability, and overall value of the products. Suitable processes for manufacturing feed rations are disclosed in Feed Manufacturing Technology IV (1994, American Feed Industry Association) and incorporated herein in its entirety.

As discussed herein, specific oil levels can be achieved in the extracted meal by altering processing conditions. The protein, amino acid, and oil levels of the present extracted meal cannot be achieved in steam-flaked normal corn, and steam-flaked high oil corn may have too much oil, which could adversely affect ruminant animal health.

Many types of animal feed rations can be developed using extracted corn meal of the present type. Types of animal feed rations using extracted corn meal are described in U.S. Pat. No. 6,648,930 at col 15, incorporated herein by reference. Human food can also be developed using extracted corn meal of the present type.

Combined LOF and extracted corn meal of the present invention can be used as an ingredient in aquaculture feed products.

One advantage of combined LOF and extracted corn meal over corn dry-milled corn products is the improved protein content and quality, since the oil has been substantially removed from the kernel resulting in a meal product in which the protein has been concentrated. This product may be used in poultry feed. Because the meal is obtained from all portions of the kernel, including the embryo, the proteins are generally of higher quality and quantity than would be found in extracted corn grits.

Combined extracted corn meal and lower oil fractions produced by the method of the present invention are also useful for fermentation-based production of compounds, such as, for example, butanol, ethanol, lactic acid, citric acid, and vitamins. Solvent extracted corn meal and/or the lower oil fraction can be hydrolyzed to provide soluble sugars. The meal/lower oil fraction serves as a carbon and nitrogen source for bacterial, fungal, or yeast cultures. Biotin and other vitamins can be produced through the cultivation of microorganisms. Organisms can include Pseudomonas mutabilis (ATCC 31014), Corynebacterium primorioxydans (ATCC 31015), Arthrobacter species, Gibberella species, Penicillium species, or combinations thereof.

Nutrients used in the cultivation of these and other microorganisms include, for example, starch, glucose, alcohols, ketones, and as a nitrogen source, peptone, corn steep liquor, soybean powder, ammonium chloride, ammonium sulfate, ammonium nitrate, extracted corn meal, or urea. Various salts and trace elements may also be included in media for the culture of microorganisms. The pH of the culture medium is about 4 to about 9, preferably about 6 to about 8, and most preferably about 7 for bacterial species. The pH is about 5 to about 7 for mold or yeast. During cultivation, temperatures are kept between 10° C. to 100° C., preferably between 20° C. to 80° C., more preferably between about 20° C. to 40° C., and most preferably about 25° C.

Biotin production is described in U.S. Pat. No. 3,859,167, incorporated herein by reference. Cis-tetrahydro-2-oxo-4-n-pentyl-thieno[3,4-d]imidazoline is added to a culture medium containing solvent extracted corn meal and other appropriate identified ingredients in combination with a microbial species capable of forming biotin. In general, the microorganism is cultivated for 1 to 10 days, preferably 1 to 8 days, and more preferably 2 to 7 days, after which time biotin is separated and purified. In one embodiment, to purify biotin, cells are removed from the culture medium, the filtrate is absorbed on activated charcoal, and purified with an ion exchange column. Alternative methods of purification are also used such as crystallization by adjusting the pH of the biotin-contained solution to near its isoelectric point.

The extracted corn meal and/or the lower oil fraction, or a combination thereof produced by the present invention can also be further processed to produce biodegradable materials. For instance, the meal or lower oil fraction of the present invention may be incorporated as a thermoplasticising agent. The meal or lower oil fraction of the present invention may be included in the methods described in U.S. Pat. No. 5,320,669, which is incorporated herein by reference. The thermoplastic material is prepared using solvent extracted corn meal, or a lower oil fraction as obtained from the process described herein. In one embodiment, the biodegradable thermoplastic composition prepared using the meal or lower oil fraction of the present invention is treated with an organic solvent, and optionally a cross-linking agent, to link together the starch and protein of the extracted corn grain. The cross-linking agent referred to herein may be any compound capable of linking the starch and the protein, such as, for example, an aldehyde, an acid anhydride, or an epoxide. The compositions so formed using the meal and/or lower oil fraction of the present invention can be used to make extruded or molded articles that are biodegradable, water-resistant, and/or have a high level of physical strength. Paper products may also comprise extracted corn meal produced by the present invention, a lower oil fraction produced by the present invention, or a combination thereof.

Blended products comprising the extracted corn meal and one or more other oilseed meals are made by one combining the extracted corn meal with extracted or non-extracted other oilseed meal to form a blended meal. At any time during these processes, additional components can be added to the blended meals to form a blended product.

The extracted corn meal can also be used in foodstuffs such as snack food, chips, food binding agents, food supplements, nutritional food bars, multivitamin-supplements, blended food products, breads, fermentation feedstock, breakfast cereals, thickened food products such canned fruit fillings, puffed or extruded foods, and porridge.

When used in edible products for humans or animals, the extracted corn meal and/or lower oil fraction can be combined with other components such as other meal, other oilseed meal, grain, other corn, sorghum, wheat, wheat milled byproducts, barley, tapioca, corn gluten meal, corn gluten feed, bakery byproduct, full fat rice bran, and rice hull.

The extracted corn meal and/or lower oil fraction can also be used as a raw material for production of corn protein isolates, for fermentation, for further chemical processing, in addition enzymes, such as amylases and proteases, can be added to the meal to help facilitate the breakdown of starch and proteins.

The extracted corn meal is optionally subjected to conventional methods of separating the starch and protein components. Such methods include, for example, dry milling, wet milling, high pressure pumping, or cryogenic processes. These and other suitable processes are disclosed in Watson, S. A. & P. E. Ramstad, ed. (1987, Corn: Chemistry and Technology, Ch. 11 and 12, American Association of Cereal Chemist, Inc., St. Paul, Minn.), the disclosure of which is hereby incorporated by reference. Due to the prior removal of oil from the corn meal, the starch, and protein components of the extracted corn meal are separated from other components more easily than they would be if the corn oil were not extracted.

Several important quality parameters for the extracted meal and lower oil fraction include the fat, starch, protein, and moisture content. Methods for evaluating quality parameters of oilseed meals are disclosed in the AOCS methods, the relevant disclosure of which is hereby incorporated by reference. These methods can also be applied to the extracted corn meal and lower oil fraction prepared as described herein.

Starting with a single corn type (e.g., 12 wt. % oil and 9 wt. % protein), more than one corn meal type can be made to meet certain nutritional requirements. The significance of this flexibility relates to the nutrient density within feed products and to dietary requirements of animals. One significant advantage of the use of this type of high oil corn and extraction process is that an extracted corn meal can be made to have a specific oil level depending on the extent of oil extraction. Once the oil is removed, the remaining corn meal has a nutrient density for protein, amino acids, and other nutrients not removed by the process, greater or different than normal corn grain and greater than that of the starting corn, e.g., 12 wt. % oil, 9 wt. % protein.

Corn meals derived using different methods or isolated at different times are compared by normalizing the meals to a common moisture content. The moisture content of an oilseed protein concentrate, such as a corn meal or whole corn, is determined using AOCS method Ba 2b-82. The crude fiber content of corn meal is determined using AOCS method Ba 6-84. AOCS method Ba 6-84 is useful for grains, meals, flours, feeds, and all fiber bearing material from which the fat can be extracted leaving a workable residue. Crude protein content of corn meal is determined using AOCS method Ba 4e-93. The starch content of corn meal is determined using AOCS method Ba 4e-93. The starch content of corn meal is determined using the Standard Analytical Methods of the Member Companies of the Corn Refiners Association Incorporated, 2d Edition, Apr. 15, 1986, method A-20 (“Corn Refiner's method A-20”).

The extracted corn meal prepared as described herein advantageously can be made to contain specific levels of oil and, in particular, specific ratios of oil to protein, of oil to carbohydrate, or of oil to protein to carbohydrate. For example, normal corn with 8 wt. % protein and 4 wt. % oil has a protein:oil ratio of 2.0, and high oil corn with 9 wt. % protein and 12 wt. % oil has a protein:oil ratio of 0.75. Meal produced by extraction to have 10.5 wt. % protein and 1.5 wt. % oil has a protein:oil ratio of 7.0. This higher ratio makes this meal type and products made from it desirable for certain applications, one example being a swine-finishing ration.

It is to be understood that the analytical methods provided herein are illustrative examples of useful methods for computing various quality parameters for the oils and meals described herein. Other suitable methods are known and may be used to compute the quality parameters disclosed and claimed herein.

The following examples are included to demonstrate specific embodiments of the present invention. It should be appreciated by those of skill in the art that the techniques disclosed in the examples which follow represent techniques discovered by the inventors to function well in the practice of the present invention, and thus can be considered to constitute exemplary modes for its practice. However, those of skill in the art should, in light of the present disclosure, appreciate that many changes can be made in the specific embodiments which are disclosed and still obtain a like or similar result without departing from the spirit and scope of the present invention.

### EXAMPLE 1

U.S. Pat. Nos. 6,313,328 and 6,388,110 describe a commercial-scale method for processing whole kernel corn grain having a total oil content of at least about 8 wt. %, including the steps of flaking corn grain and extracting a corn oil from the flaked corn grain. U.S. Pat. No. 6,610,867 describes a process for extracting corn oil to form corn meal. The process generally includes the steps of cracking whole kernel corn having a total oil content of from about 3 wt. % to about 30 wt. % and extracting a corn oil from the cracked corn grain. (Flaking is not used in this process). All components of the whole kernel (in whatever form) are subjected to the extraction step, including those components with lower oil. By contrast, in the present process, the fractionation produces a high oil fraction and a low oil fraction. The lower oil fraction bypasses the extraction process and can go directly to feed or other uses. Only the higher oil fraction is prepared for extraction and extracted. This process doubles the plant throughput with a very minimal investment.

### EXAMPLE 2

High oil corn grain 1 (LH310 (inbred, Holdens Foundation Seeds)×HOI 001, see U.S. Patent Publication Nos. 2003/018269 and 2003/0172416, incorporated herein by reference) and high oil corn grain 2 (Top Cross Blend seed corn, purchased Spring 2003, grain harvested Fall, 2003, Indiana) from storage was metered in to steam jacketed paddle mixer with a retention time of about 7 minutes. The corn was heat tempered at 90° F. The tempered corn was then conveyed to a Buhler-L apparatus, (Buhler GmbH, Germany) where the hull and softer tissue was abraded to become the lower oil fraction (“LOF”) and separated from the higher oil fraction (“HOF”). The results of the analysis of the grain, the HOF and the LOF are displayed in Table 4.

### Table 4

TABLE 4

| | Moisture | Oil | Split (wt. %) |
|---|---|---|---|
| Grain 1 | 12% | 8.6% | |
| HOF 1 | | 14.5% | 52% |
| LOF 1 | | 2.3% | 48% |
| Grain 2 | 16% | 6.7% | |
| HOF 2 | | 13.5% | 43% |
| LOF 2 | | 1.7% | 57% |

These results indicate that the oil level in over half of the corn material is reduced to less than 2.5 wt. % of the LOF, allowing this fraction to bypass the expensive extraction step.

### EXAMPLE 3

High oil corn grain 1 from Example 2 was tempered as in Example 1 prior to being fed to a Roskamp series 900 cracking mill roller (Roskamp, Waterloo, Iowa). The rollers were corrugated with 6 teeth per inch in a round bottom v design. The top roller gap was set at 2.5 mm and the bottom roller gap was set at 2.5 mm. The cracked tempered corn material was then aspirated to remove the bran. The cracked tempered de-branned corn material was then conveyed to the Buhler-L apparatus described in Example 1 for fractionation. The results of the analysis of the grain, the bran, the HOF and the LOF are displayed in Table 5.

### Table 5

TABLE 5

| | Moisture | Oil | Split (wt. %) |
|---|---|---|---|
| Grain | 12% | 8.6% | |
| Bran | | 8.9% | 13% |
| HOF | | 20.5% | 34% |
| LOF | | 3.5% | 53% |

### EXAMPLE 4

High oil corn grain 1 from Example 2 is fed to the Roskamp cracking roller mill with the settings described in Example 3 without being tempered. From the roller mill, the cracked corn material is conveyed for fractionation to the Buhler-L apparatus described in Example 2. This results in lower throughput capacity and lower oil level in the HOF than in Example 3.

### EXAMPLE 5

High oil corn grain 1 from Example 1 was fed to the Roskamp cracking roller mill described in Example 3 but with the top roller gap set at 3 mm and the bottom roller gap set at 2.5 mm. The resulting particle size distribution was 20% greater than 4 mesh (large size pieces) (19), 75% between 4 mesh and 12 mesh (medium size pieces) (19A), and 5% less than 12 mesh (small size pieces) (20). The cracked corn pieces were then screened on a Rotex screener (Rotex, Inc., Cincinnati, Ohio, Model #201GP) with a 4 mesh mill grade screen with 5.46 mm holes. The pieces that were retained on top of the screen were collected. The material which passed through the Rotex screener was conveyed to the Buhler-L apparatus described in Example 1 for fractionation. The results of the analysis of the grain, the cracked material which did not pass through the Rotex screener, the HOF and the LOF are displayed in Table 6. The large size particles were not fractionated, thus reducing costs.

### Table 6

TABLE 6

| | Moisture | Oil | Split (wt. %) |
|---|---|---|---|
| Grain | 11% | 9.6% | |
| Rotex | | 16.9% | 20% |
| retained | | | |
| HOF | | 17.16% | 31% |
| LOF | | 2.5% | 49% |

### EXAMPLE 6

The HOF from Examples 2 through 5 was collected in a surge bin and metered by a Buhler feeder DPSA (Buhler Group, address) to a Buhler homogenizer DPSD (Buhler GmbH, Germany) which added steam or water to the HOF to condition the HOF. The conditioned HOF was then fed into a Buhler Condex Expander DFEA-220 (Buhler GmbH, Germany) fitted with a 30 slot 8 mm die head to form shaped expanded HOF. The shaped, expanded HOF was cut to desired lengths by the expander. Steam was sparged into the expander barrel to give the desired plasticity to the shaped, expanded HOF. The shaped, expanded, cut lengths of HOF were cooled and subsequently solvent extracted in a hexane extraction system, such as that described in U.S. Pat. Nos. 6,388,110 and 6,313,328, incorporated herein by reference. The parameters for operation of the conditioner, sparge steam rate, expander die pressure, and expander barrel pressure were as follows:

| | |
|---|---|
| Conditioner discharge temperature | 25° C. |
| Sparge steam to expander barrel | 4% of feed rate to expander |
| Expander die pressure | 36 bar |
| Expander barrel temperature | 143° C. |

Ninety three percent of the oil from the HOF was extractable in laboratory extraction tests. Percolation and drainage were acceptable to one of ordinary skill in the art by conducting visual observation below the bed of the extractor. The draining liquid did not puddle and the intensity of the drainage was high.

### EXAMPLE 7

The pieces of cracked corn material which did not pass through the Rotex screen described in Example 4 were milled in a Fitzmill comminuter (Fitzpatrick Company, Elmhurst, Ill.) fitted with a ¼ inch screen. The ground material was mixed with the HOF material from the Buhler-L. This combined milled cracked corn and HOF was then fed to the Buhler Condex expander described in Example 5. Expandettes of acceptable extractability were formed. The extractability was measured as described in Aguilera et al., “Laboratory and Pilot Solvent Extraction of Extruded High-Oil Corn”, JAOCS, 63(2):239-243 (1986). An acceptable level of residual oil is less than about 15% of the original oil.

### EXAMPLE 8

The large size pieces of cracked corn material which did not pass through the Rotex screen described in Example 4 are heated to 70° C. The heated cracked corn material is then pressed into a 4 mm thick flake in a Roskamp (Waterloo, Iowa) model number 2862 flaking mill. The flakes are then added to the cooled, shaped expanded HOF of Example 5.

### EXAMPLE 9

The expanded higher oil fraction (9) is subjected to a supercritical carbon dioxide extraction process to remove the oil from this stream. The CO₂ can be produced locally by an ethanol-fermentation process where the feedstock material for the fermentors is one or more of the lower oil fractions (LOF (4), (lower oil cracked fraction (34)). During ethanol fermentation, for every one mole of ethanol produced, one mole of CO₂ is produced. Typically the CO₂ is vented to atmosphere and not reclaimed. In this example the CO₂ produced from the ethanol fermentation of the LOF (4) and/or lower oil cracked fraction (34) is captured and cleaned using a carbon filter to remove organic impurities. The CO₂ is then compressed and stored. The CO₂ can then be used under supercritical conditions to extract oil from the expanded HOF (9). Excess CO₂ can then be vented to atmosphere or recompressed and stored after the oil has been separated from the CO₂/oil mixture. With this system, the CO₂ used for extracting the oil is produced in one plant location thereby reducing transportation costs.

### EXAMPLE 10

Dried Distiller's Grain with Solids (“DDGS”) is a common by-product produced during dry grind ethanol fermentation. DDGS tends to be high in fiber and protein and is a good feed source for the ruminant feed industry. DDGS is high in non-digestible phosphorous, due mainly from the presence of phytate. As waste phosphorous from animal production facilities continues to be a major issue facing the animal feed industry, ways to reduce the phosphorous loading to the environment are being investigated. DDGS is especially challenging in that the DDGS contains about 3 times the phosphorous levels as the starting corn material it came from due to the typical dry grind ethanol process, for example, as described in Davis, Chippewa Valley Ethanol Company, Benson, Minn. (62^{nd} Minnesota Nuturion Conference and Minnesota Corn Growers Association Technical Symposium, Bloomington Minn., September, 2001). The dry grind ethanol process consists of utilizing whole kernel corn as the raw material for ethanol fermentation. The whole grain is ground up and the starch portion converted to sugar and the sugars are then fermented to produce ethanol and CO₂. The remaining material (e.g., oil, fiber, protein) is dried and the resulting meal is called DDGS. Since the initial phytate and phosphorous levels in the corn are not removed during the ethanol fermentation process, these components are concentrated in the remaining material, which is DDGS.

The LOF (4), and the lower oil cracked fraction (34) contain lesser amounts of the germ and fiber as compared to a whole corn kernel. The germ and fiber are where most phytate and phosphorous is located in the corn grain. Utilizing the LOF (4) and/or the lower oil cracked fraction (34) as a feedstock for ethanol fermentation provides a low phosphorous DDGS.

### EXAMPLE 11

This example details a comparison of two different feed rations: a first feed ration containing normal corn that has not been solvent extracted and a second feed ration containing extracted corn meal produced by the present invention. The feed ration containing extracted corn meal is used when lean pork meat is a desired end product. A hog finishing feed ration comprising an extracted corn meal containing less than or about 1.5 wt. % oil is prepared by providing the following ingredients in the amounts indicated in Table 7. The feed ration is generally produced by blending, mixing, and pelletting the ingredients to produce a feed product; however, one or more of these steps can be omitted in the process of preparing the feed ration. Table 7 shows a comparison of swine feed rations made using normal corn (not high oil corn) and extracted corn meal obtained from high oil corn comprising 12 wt. % oil, 9 wt. % protein, wherein the extracted corn meal has about 1.5 wt. % or less of oil (fat). Amounts are expressed on an “as is” or “as fed” moisture level.

### Table 7

TABLE 7

Swine Finishing Feed

| | Normal Corn | Extracted Corn |
|---|---|---|
| | (%) | Meal (%) |
| Ingredients | | |
| Corn | 79.98 | — |
| Extracted corn meal | — | 83.55 |
| (about 1.5% oil) | | |
| Soybean meal | 12.45 | 6.60 |
| Meat & bone meal | 6.59 | 7.22 |
| Feather meal | — | — |
| Fat | 0.10 | 1.50 |
| Salt | 0.40 | 0.70 |
| Lysine | 0.08 | 0.15 |
| Methionine | — | — |
| Premix | 0.15 | 0.15 |
| Nutrient | | |
| Crude protein, % | 15.44 | 15.78 |
| ME, kcal/kg | 3200 | 3200 |
| Crude fiber, % | 1.96 | 2.12 |
| Calcium, % | 0.85 | 0.85 |
| Phosphorus, % | 0.58 | 0.58 |
| Amino Acids, % | | |
| Arginine | 0.96 | 0.93 |
| Cyctine | 0.28 | 0.29 |
| Histidine | 0.40 | 0.42 |
| Isoleucine | 0.57 | 0.58 |
| Leucine | 1.39 | 1.49 |
| Lysine | 0.81 | 0.81 |
| Methionine | 0.26 | 0.34 |
| Phenylalanine | 0.70 | 0.72 |
| Threonine | 0.56 | 0.58 |
| Tryptophan | 0.14 | 0.14 |
| Tyrosine | 0.47 | 0.48 |
| Valine | 0.72 | 0.75 |

In Table 7, absolute values for ingredient percentages are given, however, in practice, the ingredients may include using the inclusion rates shown in other tables herein.

### EXAMPLE 12

The feed ration of this example is used to fulfill the high-energy requirements of growing birds such as broilers. A poultry broiler finishing feed ration comprising an extracted corn meal containing less than or about 4 wt. % oil (fat) is prepared by providing the following ingredients in the amounts indicated in Table 8. The feed ration is generally produced by blending, mixing, and pelletting the ingredients to produce a feed product; however, one or more of these steps can be omitted in the process of preparing the feed ration.

Table 8 shows the comparison of poultry feed rations made using normal corn (not high oil corn) and extracted corn meal obtained from high oil corn comprising 12 wt. %-oil, 9 wt. % protein, wherein the extracted corn meal has about 4 wt. % or less of oil (fat). Amounts are expressed on an “as is” or “as fed” moisture level and absolute values for ingredient percentages are given, however, in practice, the ingredients may be included using the inclusion rates shown in other tables herein.

### Table 8

TABLE 8

Growing Broiler

| | | Extracted |
|---|---|---|
| | Normal Corn (%) | Corn Meal (%) |
| Ingredients | | |
| Normal corn | 66.85 | — |
| Extracted corn meal | — | 70.86 |
| (about 4% oil) | | |
| Soybean meal | 20.96 | 16.42 |
| Meat & bone meal | 5.00 | 5.00 |
| Feather meal | 2.00 | 2.00 |
| Fat | 3.29 | 3.76 |
| Salt | 0.37 | 0.37 |
| Added Lysine | 0.13 | 0.19 |
| Added Methionine | 0.15 | 0.09 |
| Premix | 0.10 | 0.10 |
| Nutrient | | |
| Crude protein, % | 19.48 | 19.52 |
| ME, kcal/kg | 3100 | 3100 |
| Crude fiber, % | 1.97 | 2.12 |
| Calcium, % | 0.94 | 0.94 |
| Phosphorus, % | 0.63 | 0.62 |
| Amino Acids, % | | |
| Arginine | 1.27 | 1.23 |
| Cyctine | 0.38 | 0.39 |
| Histidine | 0.47 | 0.48 |
| Isoleucine | 0.78 | 0.79 |
| Leucine | 1.68 | 1.74 |
| Lysine | 1.06 | 1.06 |
| Methionine | 0.44 | 0.44 |
| Phenylalanine | 0.92 | 0.92 |
| Threonine | 0.74 | 0.75 |
| Tryptophan | 0.19 | 0.20 |
| Tyrosine | 0.61 | 0.62 |
| Valine | 0.95 | 0.96 |

### EXAMPLE 13

In this example, oil with an increased level of tocotrienol content over conventionally produced crude corn oil is described. Corn oil is solvent extracted from a higher oil fraction. The corn oil is then analyzed for tocotrienol content. Generally, increasing the extraction temperature results in an increase in the tocotrienol content of the extracted corn oil. The actual minimum and maximum values for tocotrienol content will depend upon the particular high oil corn used.

### EXAMPLE 14

This example illustrates a feed ingredient comprised of a blend of a corn meal produced by a method of the present invention and another plant-based meal such as an oilseed meal. This blended material could be in the form of simply a loose aggregate mixture of both meal types or a pelletted product. It is possible to produce both meals in proximity and blend them prior to shipment to a customer. An advantage of this approach is that varying protein and energy levels can be created in a single meal. Additional ingredients are optionally added either at the meal blending stage or at a later time. For example, an energy-intensive step in feed manufacturing involves grinding corn grain and blending it with other ingredients at a feed mill. The present blended meal generally requires less energy to produce a finished feed product than does a conventional blended meal.

Table 9 shows nutrient profiles of soybean meal (SBM), extracted corn meal (ECM), a blend of 20% SBM and 80% ECM (S20-C80), a blend of 10% SBM and 90% ECM (S10-C90), and nutrient requirements for poultry and swine diets. The poultry and swine nutrient requirements shown are in accordance with National Research Council (NRC) guidelines. The ECM was prepared according to a method of the present invention.

### Table 9

TABLE 9

| | | | | Nutrient | | Nutrient |
|---|---|---|---|---|---|---|
| | | | 20% SBM & | Needs for | 10% SBM & | Needs for |
| Parameter | SBM | ECM | 80% ECM | Poultry Diets | 90% ECM | Swine Diets |
| Crude Protein (CP) | 47.5 | 10.2 | 17.66 | 18 | 13.93 | 13.2 |
| Swine ME, kcal/kg | 3380 | 3301 | 3316.8 | | 3308.90 | 3265 |
| Poultry ME, kcal/kg | 2440 | 3133 | 2994.4 | 3200 | 3063.70 | |
| Crude Fat, % | 3 | 4 | 3.8 | | 3.90 | |
| Neutral Detergent Fiber, % | 8.9 | 11.3 | 10.82 | | 11.06 | |
| Acid Detergent Fiber, % | 5.4 | 2.8 | 3.32 | | 3.06 | |
| Arginine | 3.48 | 0.45 | 1.06 | 1.00 | 0.75 | 0.19 |
| Histidine | 1.28 | 0.27 | 0.47 | 0.27 | 0.37 | 0.19 |
| Isoleucine | 2.16 | 0.34 | 0.70 | 0.62 | 0.52 | 0.33 |
| Leucine | 3.66 | 1.03 | 1.56 | 0.93 | 1.29 | 0.54 |
| Lysine | 3.02 | 0.33 | 0.87 | 0.85 | 0.60 | 0.60 |
| Methionine | 0.67 | 0.25 | 0.33 | 0.32 | 0.29 | 0.16 |
| Cystine | 0.74 | 0.21 | 0.32 | 0.28 | 0.26 | 0.35 |
| Phenylalanine | 2.39 | 0.44 | 0.83 | 0.56 | 0.64 | 0.34 |
| Tyrosine | 1.82 | 0.29 | 0.60 | 0.48 | 0.44 | 0.55 |
| Threonine | 1.85 | 0.34 | 0.64 | 0.68 | 0.49 | 0.41 |
| Tryptophan | 0.65 | 0.09 | 0.20 | 0.16 | 0.15 | 0.11 |
| Valine | 2.27 | 0.45 | 0.81 | 0.70 | 0.63 | 0.40 |
| Total Essential Amino | 23.99 | 4.49 | 8.39 | 6.85 | 6.44 | 4.17 |
| Acids (EAA) | | | | | | |
| EAA/CP | 0.505 | 0.440 | 0.45 | 0.381 | 0.45 | 0.316 |

### EXAMPLE 15

The extracted oil is recovered and analyzed for vitamins, fatty acids, and micronutrients. As a control, 800 lbs. of yellow #2 corn is extracted in an identical manner, and the recovered oil was analyzed for the same components. Vitamin A and β-carotene are analyzed by a contract lab using a proprietary procedure. Alternative published procedures include Bates et al., Proc. Fla. State Hort Soc., 88:266-271 (1975). Free fatty acids are analyzed by gas chromatography (GC) using a CP88 cyanopropyl column (100 m×0.265 mm, 0.5 mm film thickness) and a flame ionization detector as described in American Oil Chemist Society (AOCS) methods Ce 1c-82, Ce 2-65, Cd 3a-94, and Cd 1c-85.

Tocopherols and tocotrienols are analyzed by high performance liquid chromatography (HPLC, Waters model number 2590) using a normal phase silica column with hexane-isopropanol as the mobile phase and detected using fluorescence detection (Waters model number 2690), according to the procedure described in AOCS Ce 8-89. Lutein is analyzed by HPLC using a C30 reverse phase column with water-acetonitrile mobile phase and detected with a UV detector.

Table 10 set forth below, presents a comparison of the expected oil composition to be obtained from high oil corn and yellow #2 corn. For comparison, the composition of oil from yellow #2 corn extracted in a corn wet milling process is also given.

### Table 10

TABLE 10

| | High Oil | | Y#2, Corn |
|---|---|---|---|
| Component | Corn | Yellow #2 | wet Milling |
| Palmitic Acid % | 11.4 | 10.7 | 10.7 |
| Stearic Acid % | 2.2 | 1.9 | 2.0 |
| Oleic Acid % | 35.6 | 25.5 | 27.5 |
| Linoleic Acid % | 48 | 58.4 | 57.1 |
| Linolenic Acid % | 0.7 | 1.2 | 1.1 |
| α-Tocotrienol (ppm) | 184 | 48 | 12 |
| α-Tocopherol (ppm) | 237 | 231 | 136 |
| Vitamin B1, mg/100 g | 0.390 | NA | 0.260 |
| Vitamin B2, mg/100 g | 0.090 | NA | 0.080 |
| Vitamin B6, mg/100 g | 0.82 | NA | 0.4 |
| Vitamin B12, mg/100 g | 0.5 | NA | 0.5 |

### EXAMPLE 1

This example sets forth a description of using the extracted corn meal of the present invention to produce biodegradable materials with improved tensile strength.

Extracted corn meal of the present invention is suspended in hexanes in a sealed container, at a 2:3 corn meal:solvent weight ratio. The mixture is allowed to stand at room temperature without mixing for about 18 hours. The organic solvent is removed from the extracted corn meal, and the extracted corn meal residue is washed during filtering with an aliquot of hexanes in a 1:1 residue:solvent weight ratio. The residue is dried in a convection oven at 50° C. for 16 hours. The dried residue is sprayed with water with mixing until the moisture content of the residue is 10.7% to 11.3%. The solvent-treated extracted corn meal composition is molded into an ASTM standard dogbone article using a compression molding press (Wabash Metal Products, Inc. Wabash, Ind.) at 5000 psi, 140° C. to 160° C. for 10 minutes. The untreated corn meal composition is likewise combined with water to a 10.7% to 11.3% water content and molded into an ASTM standard dogbone article. The articles produced with the solvent-treated extracted corn meal produced by a method of the present invention will exhibit significantly improved tensile properties as compared to non-solvent treated extracted corn meal.

Alternatively, corn meal of the present invention is separately suspended in aqueous ethanol (95%) at 1:3 weight-ratio of meal to oil, and boiled for 2 hours with reflux and mechanical stirring. The meal is filtered and the residues are washed with ethanol (1:1 residue:ethanol). The residues are dried, remoistened, and molded according to the procedure above. Tensile properties and water-absorption of the meal treated with ethanol at boiling temperature for a short 2 hour period would be similar to the meals treated at room temperature for an extended 18 hour period.

### EXAMPLE 17

This example sets forth the use of oil from high oil corn as a source of a biodiesel fuel.

In a continuous process, approximately 62 kg/hr (137 lbs/hr) of oil extracted from a higher oil fraction produced by the present invention and refined according to known industry processes, is mixed with 18 kg/hr (40 lbs/hr) of methanol in a stirred tank reaction unit. Simultaneously 0.08 kg/hr (0.1775 lbs/hr) of sodium hydroxide is added to the same stirred tank reaction unit, which operates at 20 psig and approximately 80° C. These conditions provide essentially 100% conversion of added triglycerides to fatty acids and methyl esters. The two phases of the reaction mixture are allowed to stand and separate to provide methyl esters in the upper phase, and a mixture of glycerol and approximately 10-15 wt. % residual methyl esters, methanol, and base in the lower phase. Approximately 6.4 kg/hr (14 lbs/hr) of the glycerol phase is neutralized, present methanol flashed off, and the remainder is sent to a continuously stirred reaction unit, operated at 80° C. and 320 psig. The reaction unit also contains approximately 4 wt. % Amberlyst-15 catalyst with a residence time of 2 hours and approximately 7.9 kg/hr (17.5 lbs/hr) iso-butylene is fed to the reaction unit. The biodiesel fuel is produced at approximately 66 kg/hr (145 lbs/hr) and has a kinematic viscosity and cloud-point that is greater than biodiesel without glycerol ethers present.

### EXAMPLE 18

### (A) Starch Hydrolysis

Solvent extracted corn meal of the present invention prepared as described herein is a rich source of starch for fermentation. The lower oil fraction of the present invention, or such lower oil fraction in combination with the extracted corn meal of the present invention can also be used as a source of starch for fermentation. One method to provide soluble sugars suitable for fermentation is to hydrolyze starch molecules. Several types of enzymes that can be used to convert starch into simple sugars are amylase(s), proteases, cellulase(s) (e.g., xylonase), esterase(s) (e.g., ferulase, acetylesterase), and ligninase(s). These enzymes may be used alone or in combination.

Five samples (i.e., one sample of yellow dent corn grain, two samples of high oil corn grain and two samples of extracted high oil corn meal produced by a method of the present invention) are ground to pass through a 1 mm screen using a Retsch Mill. High oil corn meal sample numbers 1 and 2, as shown in Table 16, are obtained from POS Pilot Plant Corporation (Saskatoon, Saskatchewan, Canada). Three hundred grams (300 g) of each sample is combined with 700 ml of water at 99° C.-100° C. comprising 0.5 ml α-amylase and placed in a sealed container. The pH of each mixture is adjusted to 5.9 with base. Each mixture is stirred for 45 min and additional α-amylase enzyme is added.

After an additional 45 min of incubation, the pH of each mixture is adjusted to 4.5 with acid. One-half of one milliliter (0.5 ml) glucoamylase (Optimax 7525) and 0.5 g protease (Fungal Protease 5000) are added to the sample mixtures and incubated with both enzymes at 62° C. for 22-24 h. Throughout the procedure, the degree of starch hydrolysis is monitored by HPLC (Waters 2690 Separations module) using an organic acid column (Aminex HPX-87H Ion Exclusion Column, 300 mm×7.8 mm, Bio Rad). Total nitrogen content for each sample is determined by Leco 2000 CN. Free amino nitrogen (FAN) is determined by the AOAC method (15^{th} Ed., 1990, p. 735).

### (B) Fermentation

Media for fermentations are normalized on a weight basis. Each sample comprises forty-five grams (45 g) of enzyme-treated and solvent extracted corn meal (resulting in starting dextrose concentrations of 133-233 g/L). Each sample is added to a 125 ml flask. Yeast extract is added at 1 g/L to ensure that nitrogen is not limiting. Cultures are inoculated with 10% inoculum from overnight yeast cultures (a typical Altech ethanol yeast of Saccharomyces cerevisiae) and incubations proceed for 42 h at 30° C. on a rotary shaker at 125 rpm. Dextrose consumption and ethanol production are monitored by HPLC.

### EXAMPLE 19

This example sets forth the use of solvent extracted corn meal from the current invention as a rich source of starch for the fermentative production of citric acid. The production of citric acid from de-fatted corn meal involves several steps including starch hydrolysis, fermentation, and citric acid recovery.

### (A) Starch Hydrolysis

Solvent extracted corn meal and the lower oil fraction of the present invention prepared as described herein is a rich source of starch for fermentation. One method to provide soluble sugars suitable for fermentation is to hydrolyze starch molecules. Types of enzymes that can be useful to convert the starch and protein matrix of corn meal into simple sugars suitable for fermentation include amylase(s), proteases, cellulase(s) (e.g., xylonase), esterase(s) (e.g., ferulase, acetylesterase), and ligninase(s). Six samples (i.e., one sample of yellow dent corn grain, one sample of yellow dent corn meal, two samples of high oil corn grain and two samples of extracted high oil corn meal) are ground to pass through a 1 mm screen using a Retsch Mill. Three hundred grams (300 g) of each sample are combined with 700 ml of water at 99° C.-100° C. comprising 0.5 ml α-amylase and placed in a sealed container. The pH of each mixture is adjusted to 5.9 with base. Each mixture is stirred for 45 min and additional α-amylase enzyme is added.

After an additional 45 min of incubation, the pH of each mixture is adjusted to 4.5 with acid. One-half of one milliliter (0.5 ml) glucoamylase (Optimax 7525) and 0.5 g protease (Fungal Protease 5000) are added to the sample mixtures and incubated with both enzymes at 62° C. or 22-24 h. Throughout the procedure, the degree of starch hydrolysis is monitored by HPLC (Waters 2690 Separations module) using an organic acid column (Aminex HPX-87H Ion Exclusion Column, 300 mm×7.8 mm, Bio Rad). Total nitrogen content for each sample is determined by Leco 2000 CN. Free amino nitrogen (FAN) is determined by the AOAC method (15^{th} Ed., 1990, p. 735).

### (B) Fermentation and Citric Acid Production

Once the starch from solvent extracted corn meal is suitably prepared through treatment with enzymes, the solution is filtered and demineralized according to commonly known practices. Resulting sugars are brought to a solids content of about 120 mg/l with demineralized water in a deep-tank fermentation vessel. The deep tank method is also known as the submerged process. In this method the tank is supplied with sterile air, nutrients and a carbon source, (hydrolyzed starch), and inoculated with Aspergillus niger spores. Spores of the fungus in a concentration of about 100 spores per liter of culture liquid, which corresponds to an amount of 10 to 15 g of spores per cubic meter (m³) are added to the nutrient solution and the citric acid production is carried out by the fungus. Examples of A. niger strains are ATCC 1015 described in U.S. Pat. No. 2,492,667, and DSM 5484 described in U.S. Pat. No. 5,081,025.

The incubation of the broth thus inoculated is carried out at conditions generally known and described for citric acid production, such as continued aeration and temperature control. During the fermentation process, the temperature is maintained at about 32° C. (90° F.), the pH is maintained at about 2 to 3 with sodium citrate, and sterile air is added to maintain about 50% dissolved oxygen content. Fermentation is carried out until the fermentation broth reaches a reducing sugar content of about 1 g/L, which may require several days to achieve. Two main separation processes can be used in the recovery of citric acid, the Lime-Sulfuric Acid process and the Liquid extraction process. The Lime-Sulfuric Acid method is commonly used and is familiar to those skilled in the art of citric acid production.

### EXAMPLE 20

This example describes the extraction of oil from yellow dent #2 corn (commodity field corn).

The HOF produced from Yellow dent #2 corn was expanded using a model DFEA-220 expander (Buhler GmbH, Germany) to create collets. Moisture was introduced in the form of steam into the expander barrel. The rate of steam addition ranged from 6.0 to 6.8%. The expanded HOF was cooled in a horizontal ambient air cooler that reduced the moisture content to between 10 and 13% moisture. The HOF was expanded to make it suitable for presentation to a full-scale solvent extractor.

Two truckloads of the expanded HOF were metered into a full-scale extraction process at an inclusion of 23-32% HOF. The balance was wet milled germ expeller cake. The trucks were unloaded into the wet milled germ flow over a 3.5 hr period. The combination was extracted in a shallow bed Crown Model III extractor. The extractor is sized for 1000 T/day. Table 11 shows results of different sample points during the trial:

### Table 11

TABLE 11

| | Fat % | Protein % | Moisture % | FFA % |
|---|---|---|---|---|
| Extractor Feed | 16.0-17.2 | — | 4.96-7.06 | 2.0-3.4 |
| Extractor Discharge | 0.98-1.33 | — | 7.80-8.95 | — |
| DC/DT Discharge | 2.77-4.92 | 19.02-20.21 | 10.99-12.18 | — |
| Finished Oil to Storage | — | — | — | 1.4-1.7 |

FFA = free fatty acids,
DC = dryer/cooler
DT = desolventizer toaster

Similarly, HOF is produced from yellow #2 corn. It is made into a solvent-extractable structure, solvent extracted and the extracted corn meal is used as a feedstock for fermentation.

### EXAMPLE 21

This example sets forth one embodiment of a combination of wet and dry milling techniques.

Dry milling corn provides a crude separation of the kernel components. It is typically used when high purity starch and other products are not necessary. It is generally used to produce fermentation feedstock in ethanol producing facilities, because the yeast does not need high purity feedstock. Dry milling is less capital intensive and uses less energy than wet-milling. In contrast, wet-milling provides high purity starch, protein, and oil. The use of a mechanical separation step, such as a fractionation step, prior to using one or more wet-milling techniques, allows for the production of high purity products without using a process that is as energy and capital intensive as wet milling.

In the corn wet-milling process the soaking of the grain is termed “steeping.” The steeping process of corn, generally, includes the addition of sulfur dioxide (from about 0.1 to about 0.3%) and steeping times of from about 24 to about 48 hours at temperatures between from about 45 to about 60° C. After steeping, light steep water is obtained, which contains a high percentage of the soluble parts from the corn kernels. The resulting steeped corn kernels are relatively softer than they were prior to steeping and at the end of the steeping process they can be separated into germs, fiber, starch and proteins.

The steeped corn is coarse ground in a coarse grinding mill in two steps to release the germ from the kernels. The germs are separated after each coarse milling step. Germs have an oil content of approximately 45-55%. The oil is usually extracted in subsequent refining steps.

The remaining coarse de-germed kernels are milled in a coarse grinding mill for the third time to disrupt the endosperm matrix and release the starch. Fibers are removed from the starch and endosperm proteins by passing the slurry over a series of screens.

The separated fiber is then dewatered and dried. In some instances the fiber is combined with steep water that has been concentrated in evaporators until it reaches about 45 to about 50% dry solids. The dried mixture of fiber and steepwater is referred to as corn gluten feed.

The remaining starch protein mixture is thickened and separated using a series of centrifuges. In the mill-stream thickener (MST) centrifuge, the feed density is increased to improve separation of starch and endosperm protein (gluten). The overflow from the MST is sent to the steep house for use as steep water. The underflow from the MST is sent to the primary centrifuge (primary separation step). In the primary separation step the gluten proteins are partially separated from the starch. The overflow from the primary centrifugation step is the light gluten stream. The primary underflow is sent to starch washing to purify the starch. Overflow from the starch wash step is thickened in the clarifying centrifuge. The clarifier underflow is returned to the primary centrifuge feed tank. The clarifier overflow is used for primary centrifuge wash water and fiber wash water.

The light gluten stream, containing about 5% dry solids, is concentrated in the gluten thickener centrifuge. The overflow is used for fiber and germ washing. The underflow, referred to as heavy gluten contains from about 10 to about 20% of dry substance, mainly insoluble proteins (about 64% on dry base) and from about 10 to about 25% of starch (on dry base). The suspended solids in the heavy gluten are separated from the process water with rotary vacuum filters. The gluten cake that discharges from the filters contains about 55 to about 65% water. The process water (sometimes referred to as gluten filtrate) that was separated from the gluten cake is returned to the gluten thickener feed tank. The gluten cake is dried to a moisture content of about 10 to about 12%, and is referred to as corn gluten meal. The HOF and the LOF generated in the fractionation step(s) shown in FIGS. 1 and 2 can be used in a wet-milling process, however, they will be inputted into the wet-milling process at different points.

LOF has already had a significant amount of the germ removed in the fractionation step. Therefore, the LOF can be input into the wet-milling process during the steeping step (steeping can be either batch or continuous), but it is expected that the LOF will require a much shorter steep time, and less SO₂ and possibly eliminating the need for SO₂. The steep time can additionally be made shorter by incorporating the use of ultrasound, mixing, and the like into the process.

In another embodiment, the steeping process can be skipped by dry grinding the LOF and introducing the ground LOF into the third grind or fiber wash. In embodiments where the ground LOF is being integrated into an existing running wet-mill, it is expected that the concentration of the SO₂ at the third grind will be sufficient to facilitate the separation of the protein from the starch.

In some embodiments, it may be desirable to add organic acids to facilitate the separation of the LOF. Organic acids that may be useful for this process included for example lactic acid, citric acid, and the like.

The HOF, e.g. after expansion, is mixed, as in Example 20, with germ recovered from wet-milling and the products are combined prior to extraction.

One of ordinary skill in the art will appreciate that any product that can be made in a traditional wet-mill can be made by a mill that incorporates the use of HOF or LOF into the process. For example, such products include crude oil, fermentation feedstocks, high fructose corn syrup, corn syrup, sweeteners, corn gluten feed, corn gluten meal, starch, extracted meal, animal feed, fertilizer, and the like.

### EXAMPLE 22

This example compares the recovery of oil between using one and two stage fractionation.

Two batches of approximately 3 tons each of Mavera™ high value corn (Renessen S. R. L., Argentina) were fed to a Buhler-L apparatus, one batch after tempering with 1.6% water and one batch without tempering, to form HOF and LOF. The larger pieces of LOF that were discharged from the Buhler-L were sifted across a 6000 micron screen MPAD Pansifter. The material that went through the screen became small LOF. The material that was retained on the screen (retained LOF) was fed to a Buhler-L apparatus for processing. The resulting second stage HOF from this step was added to the HOF from the first stage fractionation. Likewise, the LOF from the second stage fractionation step was combined with the small LOF stream. By adding the second fractionation stage, the percentage of the recovered oil from the HOF fractions of the grain increased over a single stage fractionation. Table 12 compares the results from 1 stage fractionation (no tempering), 2 stage fractionation (no tempering) and 2 stage fractionation, tempering with 1.6% water.

### Table 12

TABLE 12

| | % fat - | % | % fat - | % | Recovery |
|---|---|---|---|---|---|
| | LOF | LOF/feed | HOF | HOF/feed | of oil |
| 1 stage | 3.4% | 80% | 17.8% | 20% | 56% |
| (no tempering) | | | | | |
| 2 stages | 1.4% | 64% | 16% | 36% | 89% |
| (no tempering) | | | | | |
| 2 stages- | 1.0% | 60% | 14% | 40% | 87% |
| tempering | | | | | |
| with 1.6% water | | | | | |

### EXAMPLE 23

This example illustrates an increase in mill efficiency when producing pelleted feeds by using the a combined meal comprising solvent extracted HOF and LOF (The “Enhanced Meal) of the current invention.

Feed pellets formulated for a broiler diet were produced using Enhanced Meal, with increasing levels of added fat, and compared to pellets formulated to the same diet using standard yellow #2 corn. For all diets the Enhanced Meal was substituted at an equal weight basis for the ground yellow #2 corn. The formulations for the above treatments are described in Table 13.

### Table 13

TABLE 13

| | Treatment | | | | |
|---|---|---|---|---|---|
| | | Enhanced | Enhanced | Enhanced | Enhanced |
| | | Meal | Meal | Meal | Meal |
| | Control | 0% added | 1.5% added | 2.0% added | 2.5% added |
| INGREDIENT | Y#2 Corn | fat | fat | fat | fat |
| Corn/Enhanced | 65.00% | 65.00% | 65.00% | 65.00% | 65.00% |
| Meal | | | | | |
| Soybean meal | 31.50 | 31.50 | 31.50 | 31.50 | 31.50 |
| Soybean oil | 1.50 | 0.00 | 1.50 | 2.00 | 2.50 |
| Limestone | 1.34 | 1.34 | 1.34 | 1.34 | 1.34 |
| Salt | 0.28 | 0.28 | 0.28 | 0.28 | 0.28 |
| DL-methionine | 0.08 | 0.08 | 0.08 | 0.08 | 0.08 |
| Poultry Premix** | 0.25 | 0.25 | 0.25 | 0.25 | 0.25 |

**Premix contains supplement vitamins and trace minerals which meets or exceeds those required for normal growth in poultry as outlined in National Research Council Poultry NRC, 1994

Additionally, pellets for companion animal and aquaculture feeds are generated using either Enhanced Meal or standard yellow #2 corn. Examples of these formulations are shown in Tables 14-15.

### Table 14

TABLE 14

| Ingredient | % |
|---|---|
| Yellow #2 corn/Enhanced Meal | 17 |
| Rice Bran | 4.5 |
| Soybean Meal | 32 |
| Salt | 0.5 |
| Calcium Carbonate | 0.74 |
| Fish Meal | 10 |
| Mono-Dical Phosphate | 2 |
| DL-methionine | 0.26 |
| Fat | 1.5 |
| Wheat Middlings | 25 |
| Poultry Meal | 1.8 |
| Feather Meal | 4 |
| Vitamin Premix** | 0.3 |
| Trace Mineral Premix** | 0.4 |
| Total | 100 |

**Premix contains supplement vitamins and trace minerals which meets or exceeds those required for normal growth in poultry as outlined in National Research Council Poultry NRC, 1994

### Table 15

TABLE 15

| Ingredient | % |
|---|---|
| Yellow #2 corn/Enhanced Meal | 56.12 |
| Wheat Middlings | 5 |
| Meat and Bone Meal | 15 |
| Salt | 0.5 |
| Mono-dical | 2.4 |
| Corn Gluten Meal | 9.1 |
| Choline CHL-60 | 0.08 |
| Brewer's Rice | 10 |
| Vitamin Premix** | 0.2 |
| Trace Mineral Premix** | 0.1 |
| Fat | 1.5 |
| TOTAL | 100 |

**Premix contains supplement vitamins and trace minerals which meets or exceeds those required for normal growth in poultry as outlined in National Research Council Poultry NRC, 1994

The Enhanced Meal was prepared by pelleting a combination of solvent extracted HOF with LOF. was received in bulk as ¼ inch pellets, having a loose bulk density of 54 Kg/hl and 32% fines passing through a #14 screen. The proximate analysis for this product is shown in Table 16. The results show that the Enhanced Meal was 2.0% lower in fat content as compared to yellow #2 corn of an equivalent weight.

### Table 16

TABLE 16

| | |
|---|---|
| Ash, % | 1.80 |
| Moisture, % | 11.51 |
| Fat Acid Hydrolysis, % | 3.21 |
| Ether Extract Fat, % | 1.55 |
| Protein, % | 8.68 |
| ADF, % | 2.43 |
| NDF, % | 9.73 |
| Crude Fiber, % | 2.00 |

The yellow #2 corn grain and Enhanced Meal were ground prior to pelleting using a Jacobson model P-240, 30 hp hammermill, equipped with new hammers and an 8/64″ screen. The results showed that at the same motor load, the Enhanced Meal resulted in an increased throughput (lbs/hr) of 42% and a decrease in energy consumption (Kwh/T) of about 30% per ton processed. These results indicate that there would be a significant cost savings to the mill by using Enhanced Meal as compared to yellow #2 corn.

Feed pellets comprising Enhanced Meal or yellow #2 corn grain were generated using methods well known in the art (see for example Gilpin et al., Applied Engineering in Agriculture 18(3): 331-338 (2002)). The pellets were formed using a CPM Master HD model (California Pellet Mill Company, Crawfordsville, Ind.) pellet mill, which was equipped with a 5/32″×1.25″ die. Conditioning temperature was held constant at 180° F. (80° C.). The feed screw rate was held constant at 8.8 revolutions per minute (rpm). A recording volt/amp meter was attached to the pellet mill drive motor and voltage and amp loads were recorded and averaged across the treatment run.

The pellet quality was determined as described in Appendix E (pp. 551-552) and Appendix F (p. 558), Feed Manufacturing Technology IV, American Feed Industry Association (1994). A modified, more rigorous version of the aforementioned standard PDI test was done by including five ½″ hex nuts to the tumble chamber (modified PDI).

The results indicate that all pellet formulations, regardless of oil inclusion level, resulted in substantially improved pellet quality compared to the control. For example, results for the modified PDI test showed values of greater than 90% for all of the Enhanced Meal treatments as compared to 83% for the control of yellow #2 corn (Table 17). Previous experiments had shown that pellets formed with equivalent levels of oil, for example shell corn, had modified PDI ratings no greater than 85%.

### Table 17

TABLE 17

| | | | Enhanced | Enhanced | Enhanced |
|---|---|---|---|---|---|
| | | Enhanced | Meal | Meal | Meal |
| | Control | Meal 0% | 1.5% | 2.0% | 2.5% |
| | (Y#2) | added Fat | added Fat | added Fat | added Fat |
| Standard PDI | 87.55 | 97.40 | 95.44 | 94.72 | 92.88 |
| Modified PDI | 83.26 | 96.80 | 94.52 | 93.30 | 90.80 |

The results in terms of production rate (lb/hr) indicate that there were no statistical differences among treatments (Table 18). There was, however, a substantial difference in the relative energy usage (Kwh/T) between the Enhanced Meal with no added fat and all other diets (Table 19). This result was expected as the lack of a lubricating effect from the low oil content of the Enhanced Meal would result in increased friction in the die and rolls.

The results of these studies indicate that the Enhanced Meal with 2.5% added fat level would be the best formulation for broiler feed pellets in terms of production rate, energy usage and pellet quality. Similar results are seen with aquaculture and companion animal feeds.

### Table 18

TABLE 18

| | Treatment | |
|---|---|---|
| | Control (Y#2 corn) | Enhanced Meal |
| lbs/hr | 3760 | 5326 |
| T/hr | 1.88 | 2.66 |
| Kwh/T | 4.90 | 3.41 |
| T/Kwh | 408 | 586 |
| Particle size (microns) | 606 | 638 |
| Particle (standard deviation) | 2.12 | 2.08 |

### Table 19

TABLE 19

| | | Treatment | Enhanced | Enhanced | |
|---|---|---|---|---|---|
| | | Enhanced | Meal | Meal | Enhanced |
| | | Meal 0% | 1.5% | 2.0% | Meal 2.5% |
| Response | #2 YC | added Fat | added Fat | added Fat | added Fat |
| Production | 1889.59 | 1848.42 | 1853.93 | 1823.33 | 1890.16 |
| Rate (lb/hr) | | | | | |
| Kwh/T | 11.3 | 13.0 | 11.8 | 11.83 | 11.37 |
| Density | 52.2 | 55.4 | 54.9 | 53.09 | 52.31 |
| (Kg/hL) | | | | | |

Unless otherwise defined, all technical and scientific terms and abbreviations used herein have the same meaning as commonly understood by one of ordinary skill in the art to which this invention pertains. Although methods and materials similar or equivalent to those described herein can be used in the practice of the present invention, suitable methods and materials are described below without intending that any such methods and materials limit the invention described herein. All patents publications and official analytical methods referred to herein are incorporated by reference in their entirety. Additional features and advantages of the present invention will be apparent from the following description of illustrative embodiments of the present invention and from the claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the present invention.

Preferred embodiments of the present invention are described herein, including the best mode known to the inventors for carrying out the present invention. Of course, variations of those preferred embodiments will become apparent to those of ordinary skill in the art upon reading the foregoing description. The inventors expect skilled artisans to employ such variations as appropriate, and the inventors intend for the present invention to be practiced otherwise than as specifically described herein. Accordingly, the present invention includes all modifications and equivalents of the subject matter recited in the claims appended hereto as permitted by applicable law. Moreover, any combination of the above-described elements in all possible variations thereof is encompassed by the present invention unless otherwise indicated herein or otherwise clearly contradicted by context.

1. A method of dividing a whole corn kernel of high oil corn comprising:
fractionating a whole corn kernel of high oil corn having a range of moisture from about 8 wt. % to about 22 wt. %, and further having an endosperm component and a germ component, into a higher oil fraction and a lower oil fraction, wherein, the higher oil fraction has an oil concentration greater than that of the corn kernel and the lower oil fraction has an oil concentration less than that of the corn kernel.

2. The method of claim 1, wherein fractionating comprises contacting the whole corn kernel with an abrasive screen to separate at least a portion of the germ component of the corn kernel from at least a portion of the remainder of the corn kernel.

3. The method of claim 2, wherein fractionating comprises subjecting the whole corn kernel to a Buhler L machine, a Satake debranner, or other means for contacting the corn kernel with a device to remove at least a portion of the germ component of the corn kernel from at least a portion of the remainder of the corn kernel.

4. The method of claim 1, further comprising separating the lower oil fraction into larger and smaller pieces of lower oil fraction and fractionating the larger pieces of the lower oil fraction into a second stage higher oil fraction and a second stage lower oil fraction; and, optionally, combining the second stage higher oil fraction and the higher oil fraction; and, optionally, combining the second stage lower oil fraction and the lower oil fraction.

5. A method of dividing a whole corn kernel of high oil corn comprising:
a) cracking into at least two differing sized pieces of cracked corn, a whole corn kernel of high oil corn having a range of moisture from about 8 wt. % to about 22 wt. %, and further having an endosperm component and a germ component; and, optionally, further separating a portion of the cracked corn pieces into at least two fractions according to their size; and
b) fractionating the cracked corn or optionally separated larger pieces of cracked corn into a higher oil fraction and a lower oil fraction, wherein, the higher oil fraction has an oil concentration greater than that of the corn kernel and the lower oil fraction has an oil concentration less than that of the corn kernel.

6. The method of claim 5, further comprising separating the lower oil fraction into larger and smaller cracked pieces of lower oil fraction and fractionating the larger pieces of the cracked lower oil fraction into a second stage cracked higher oil fraction and a second stage cracked lower oil fraction; and optionally, combining the second stage cracked higher oil fraction and the higher oil fraction; and, optionally, combining the second stage cracked lower oil fraction and the lower oil fraction.

7. The method of claim 1, further comprising tempering the corn kernel at a temperature and for a time sufficient to increase the differential hardness between the germ component and the remainder of the corn kernel.

8. The method of claim 5, further comprising tempering the cracked corn pieces at a temperature and for a time sufficient to increase the differential hardness between the germ component and the remainder of the cracked corn pieces.

9. The method of claim 1, further comprising extracting oil from a portion of the higher oil fraction, the second stage higher oil fraction, the cracked higher oil fraction, or the larger pieces of the cracked pieces of the second stage oil fraction to produce an extracted corn oil and an extracted corn meal.

10. The method of claim 9, further comprising forming a portion of the higher oil fraction, the second stage higher oil fraction, the cracked higher oil fraction, or the larger pieces of the cracked pieces of the second stage oil fraction into a solvent-extractable structure.

11. The method of claim 10, wherein forming comprises one or more of extruding, expanding, expelling, pelleting or enzymatic treatment.

12. The method of claim 11, further comprising solvent extraction of oil from a portion of the solvent-extractable structure to produce an extracted corn oil and an extracted corn meal.

13. The method of claim 11, further comprising desolventizing a portion of the extracted corn meal or extracted corn oil.

14. The method of claim 5, further comprising aspirating a portion of the smaller size pieces of cracked corn to remove bran.

15. The method of claim 5, comprising flaking a portion of the larger size pieces of cracked corn to produce flaked, cracked corn.

16. The method of claim 5, further comprising grinding a portion of the larger size pieces of cracked corn to produce ground, cracked corn.

17. The method of claim 15, further comprising solvent extraction of oil from a portion of the flaked, cracked corn material to produce an extracted corn oil and an extracted corn meal.

18. The method of claim 16, further comprising solvent extraction of oil from a portion of the ground, cracked corn material to produce an extracted corn oil and an extracted corn meal.

19. The method of claim 16, further comprising forming a portion of the ground cracked corn material into a solvent-extractable structure.

20. The method of claim 16, further comprising combining a portion of the ground cracked corn and a portion of the higher oil fraction form a first combination material, and, optionally, forming a portion of the first combination material into a solvent extractable structure.

21. The method of claim 19, wherein forming into a solvent extractable structure comprises one or more of extruding, expanding, expelling, pelleting, or enzymatic treatment.

22. The method of claim 21, further comprising solvent extraction of oil from a portion of the solvent-extractable structure of the first combination material to produce an extracted corn oil and an extracted corn meal.

23. The method of claim 20, comprising flaking a portion of the larger size pieces of cracked corn to product flaked, cracked corn and further comprising combining a portion of the first combination material with a portion of the flaked, cracked corn material produced by the method of claim 15, to form a second combination material.

24. The method of claim 23, further comprising solvent extraction of oil from a portion of the second combination material to produce an extracted corn oil and an extracted corn meal.

25. The method of claim 15, further comprising combining a portion of the flaked, cracked corn and a portion of the higher oil fraction.

26. The method of claim 25, further comprising solvent extraction of oil from the third combination material to produce an extracted corn oil and an extracted corn meal.

27. The method of claim 9, further comprising desolventizing a portion of the extracted corn meal.

28. The method of claim 1, further comprising using a portion of the lower oil fraction as a feedstock for fermentation, wet corn milling, food pet food or other process.

29. The method of claim 27, further comprising using a portion of the desolventized, extracted meal as a feedstock for fermentation, wet corn milling, food, pet food, or other process.

30. The method of claim 12, wherein the solvent comprises carbon dioxide from a fermentation process.

31. The method of claim 1, further comprising using a portion of the higher oil fraction as a feedstock for fermentation, food, pet food, or other process.

32. The method of claim 14, further comprising using a portion of removed bran as a feedstock for extraction.

33. The method of claim 32, further comprising extracting a portion of phytosterols from the bran feedstock.

34. (canceled)

35. A method of dividing a corn kernel comprising:
fractionating at least one corn kernel having a range of moisture from about 8 wt. % to about 22 wt. %, and further having an endosperm component and a germ component, into a higher oil fraction and a lower oil fraction, wherein the lower oil fraction has an oil concentration less than that of the corn kernel and the higher oil fraction has an oil concentration greater than that of the corn kernel, and wherein less than 50% of the germ in the higher oil fraction is intact.

36. The method of claim 35, further comprising using at least a portion of the lower oil fraction for one or more processes selected from the group consisting of fermentation, wet-milling, animal feed production, sweetener production, and starch production.

37. The method of claim 35, further comprising using at least a portion of the higher oil fraction for one or more processes selected from the group consisting of; fermentation, oil production, wet-milling, animal feed production, sweetener production, and starch production.

38. The method according to claim 35, wherein the fractionating is accomplished using a mechanical debranning machine.

39. The method according to claim 35, further comprising forming a portion of the higher oil fraction into a solvent-extractable structure.

40. The method of claim 39, wherein forming comprises one or more of extruding, expanding, expelling, pelleting or enzymatic treatment.

41. The method of claim 40, further comprising solvent extraction of oil from a portion of the solvent-extractable structure to produce an extracted corn oil and an extracted corn meal.

42. The method of claim 41, further comprising fermenting a portion of the extracted corn meal.

43. An extracted meal product produced by the method of claim 1.

44. A lower or higher oil fraction produced by the method of claim 1.

45. A solvent extractable structure produced by the method of claim 1.

46. The first combination material of claim 20.

47. The second combination material of claim 23.

48. The third combination material of claim 25.

49. The aspirated bran of claim 14.

50. The extracted corn oil. produced by the method of claim 1.

51. The desolventized extracted meal. produced by the method of claim 1.

52. The desolventized extracted corn oil. produced by the method of claim 1.

53. A human food or animal feed comprising an extracted meal, extracted oil, or lower oil fraction. produced by the method of claim 1.

54. A biodiesel comprising the extracted corn oil. produced by the method of claim 1.

55. A fermentation-based product produced from a product selected from the group consisting of an extracted meal product, a lower oil fraction, and a higher oil fraction, wherein said product is produced by the method of claim 1.

56. The animal feed of claim 53, wherein the animal feed is a swine feed, poultry feed, poultry layer feed, cattle feed, equine feed, dairy cattle, aquaculture feed, or pet feed.

57. The animal feed of claim 53, wherein the animal feed has been pelleted to form a pelleted meal.

58. A corn oil-containing product comprising the corn oil produced by the method of claim 1, wherein the corn oil comprises: oil extracted from at least the germ and endosperm of the corn; and at least one other component extracted from one or more of the endosperm, tip cap or pericarp of the corn.

59. The corn oil-containing product of claim 58, wherein the corn oil comprises: oil extracted from the germ and the endosperm of the corn; and at least one component selected from one or more of the endosperm, tip cap or pericarp of the corn, wherein the component is selected from the group consisting of a carotene, pigment, tocotrienol, tocopherol, antioxidant, fat soluble vitamin and sterol.

60. The corn oil-containing product of claim 58, further comprising one or more materials selected from the group consisting of conventional corn oil, soy oil, canola oil, olive oil, palm oil, sunflower oil, safflower oil, antioxidant, flavoring, hydrogenated oil, partially hydrogenated oil, and animal fat.

61. The method of claim 9, wherein the method further comprises the step of including the corn oil in a raw material stream of an oil refining process.

62. The method of claim 9, further comprising refining the corn oil.

63. A biodegradable product made from a material selected from the group consisting of extracted corn meal, the lower oil fraction and a combination of extracted corn mean and lower oil fraction, wherein said material is produced by the method of claim 1.

64. The biodegradable product of claim 63, wherein the extracted corn meal is treated with an organic solvent.

65. The biodegradable product of claim 64, wherein the extracted corn meal is further treated with a cross-linking agent.

66. A paper product comprising a material selected from the group consisting of the extracted corn meal claim 1, the lower oil fraction, and a combination of extracted corn meal and lower oil fraction, wherein said material is produced by the method of claim 1.

67. A method for producing fermentation-based products comprising:
a) combining an enzyme, water, and a material selected from the group consisting of the extracted corn meal, the lower oil fraction and a combination of extracted corn meal and lower oil fraction, wherein said material is produced by the method of claim 1.
b) incubating the combination; and
c) mixing the combination with a micro-organism capable of fermenting a carbon source to produce a fermentation-based product.

68. The method of claim 11, wherein the extraction is accomplished by extracting corn oil using a continuous solvent extraction process.

69. A method of forming an extracted blended meal comprising an extracted meal obtained from corn and one or more extracted other oilseed meals, the method comprising:
a) combining an extracted meal with one or more extracted other oilseed meals to form a blended meal, wherein the extracted meal has been prepared by fractionating a corn material into a higher oil fraction and a lower oil fraction,
b) separating at least a portion of the higher oil fraction from the lower oil fraction,
c) forming the higher oil fraction into a solvent extractable structure, and
d) extracting the oil to produce the extracted meal.

70. The method of claim 69, further comprising adding the lower oil fraction to the extracted meal or the extracted blended meal.

71. A method according to claim 69, wherein the one or more other oilseed is selected from the group consisting of soybean, canola seed, sunflower seed, oilseed rape, and cotton seed.

72. The method of claim 5, further comprising extracting oil from a portion of the higher oil fraction, the second stage higher oil fraction, the cracked higher oil fraction, or the larger pieces of the cracked pieces of the second stage oil fraction to produce an extracted corn oil and an extracted corn meal.

73. The method of claim 72, further comprising forming a portion of the higher oil fraction, the second stage higher oil fraction, the cracked higher oil fraction, or the larger pieces of the cracked pieces of the second stage oil fraction into a solvent-extractable structure.

74. The method of claim 73, wherein forming comprises one or more of extruding, expanding, expelling, pelleting or enzymatic treatment.

75. The method of claim 74, further comprising solvent extraction of oil from a portion of the solvent-extractable structure to produce an extracted corn oil and an extracted corn meal.

76. The method of claim 74, further comprising desolventizing a portion of the extracted corn meal or extracted corn oil.

77. The method of any one of claim 5, further comprising using a portion of the lower oil fraction as a feedstock for fermentation, wet corn milling, food pet food or other process.

78. The method of claim 75, wherein the solvent comprises carbon dioxide from a fermentation process.

79. The method of any of claim 5, further comprising using a portion of the higher oil fraction as a feedstock for fermentation, food, pet food, or other process.

80. An extracted meal product produced by the method of claim 5.

81. An extracted meal product produced by the method of claim 35.

82. A lower or higher oil fraction produced by the method of claim 5.

83. A lower or higher oil fraction produced by the method of claim 35.

84. A solvent extractable structure produced by the method of claim 5.

85. A solvent extractable structure produced by the method of claim 35.

86. The extracted corn oil produced by the method of claim 5.

87. The extracted corn oil produced by the method of claim 35.

88. The desolventized extracted meal produced by the method of claim 5.

89. The desolventized extracted meal produced by the method of claim 35.

90. A human food or animal feed comprising an extracted meal, extracted oil, or lower oil fraction produced by the method of claim 5.

91. A human food or animal feed comprising an extracted meal, extracted oil, or lower oil fraction produced by the method of claim 35.

92. A biodiesel comprising the extracted corn oil produced by the method of claim 5.

93. A biodiesel comprising the extracted corn oil produced by the method of claim 35.

94. A fermentation-based product produced from a material selected from the group consisting of an extracted meal product, a lower oil fraction, and a higher oil fraction, wherein said material is produced by the method of claim 5.

95. A fermentation-based product produced from a material selected from the group consisting of an extracted meal product, a lower oil fraction, and a higher oil fraction, wherein said material is produced by the method of claim 35.

96. The animal feed of claim 90, wherein the animal feed is a swine feed, poultry feed, poultry layer feed, cattle feed, equine feed, dairy cattle, aquaculture feed, or pet feed.

97. The animal feed of claim 91, wherein the animal feed is a swine feed, poultry feed, poultry layer feed, cattle feed, equine feed, dairy cattle, aquaculture feed, or pet feed.

98. The animal feed of claim 90, wherein the animal feed has been pelleted to form a pelleted meal.

99. The animal feed of claim 91, wherein the animal feed has been pelleted to form a pelleted meal.

100. A method of improving pellet quality or improving efficiency of producing pelleted animal feeds comprising substituting the pelleted meal of claim 57 for yellow #2 corn.

101. A method of improving pellet quality or improving efficiency of producing pelleted animal feeds comprising substituting the pelleted meal of claim 100 for yellow #2 corn.

102. A method of improving pellet quality or improving efficiency of producing pelleted animal feeds comprising substituting the pelleted meal of claim 101 for yellow #2 corn.

103. A corn oil-containing product comprising the corn oil produced by the method of claim 5, wherein the corn oil comprises: oil extracted from at least the germ and endosperm of the corn; and at least one other component extracted from one or more of the endosperm, tip cap or pericarp of the corn.

104. A corn oil-containing product comprising the corn oil produced by the method of claim 35, wherein the corn oil comprises: oil extracted from at least the germ and endosperm of the corn; and at least one other component extracted from one or more of the endosperm, tip cap or pericarp of the corn.

105. The corn oil-containing product of claim 103, wherein the corn oil comprises: oil extracted from the germ and the endosperm of the corn; and at least one component selected from one or more of the endosperm, tip cap or pericarp of the corn, wherein the component is selected from the group consisting of a carotene, pigment, tocotrienol, tocopherol, antioxidant, fat soluble vitamin and sterol.

106. The corn oil-containing product of claim 104, wherein the corn oil comprises: oil extracted from the germ and the endosperm of the corn; and at least one component selected from one or more of the endosperm, tip cap or pericarp of the corn, wherein the component is selected from the group consisting of a carotene, pigment, tocotrienol, tocopherol, antioxidant, fat soluble vitamin and sterol.

107. The corn oil-containing product of claim 105, further comprising one or more materials selected from the group consisting of conventional corn oil, soy oil, canola oil, olive oil, palm oil, sunflower oil, safflower oil, antioxidant, flavoring, hydrogenated oil, partially hydrogenated oil, and animal fat.

108. The corn oil-containing product of claim 106, further comprising one or more materials selected from the group consisting of conventional corn oil, soy oil, canola oil, olive oil, palm oil, sunflower oil, safflower oil, antioxidant, flavoring, hydrogenated oil, partially hydrogenated oil, and animal fat.

109. A biodegradable product made from a material selected from the group consisting of the extracted corn meal, the lower oil fraction, and a combination of extracted corn mean and lower oil fraction, wherein said material is produced by the method of claim 5.

110. A biodegradable product made from a material selected from the group consisting of the extracted corn meal, the lower oil fraction, and a combination of extracted corn mean and lower oil fraction, wherein said material is produced by the method of claim 35.

111. The biodegradable product of claim 109, wherein the extracted corn meal is treated with an organic solvent.

112. The biodegradable product of claim 110, wherein the extracted corn meal is treated with an organic solvent.

113. The biodegradable product of claim 111, wherein the extracted corn meal is further treated with a cross-linking agent.

114. The biodegradable product of claim 112, wherein the extracted corn meal is further treated with a cross-linking agent.

115. A paper product comprising a material selected from the group consisting of the extracted corn meal, the lower oil fraction, and a combination of extracted corn meal and lower oil fraction, wherein said material is produced by the method of claim 5.

116. A paper product comprising a material selected from the group consisting of the extracted corn meal, the lower oil fraction, and a combination of extracted corn meal and lower oil fraction, wherein said material is produced by the method of claim 35.
